(19) United States
(12) Patent Application Publication
(10) Pub. No.: US 2018/0110939 A1
(43) Pub. Date: Apr. 26, 2018

(54) METHOD, SYSTEM AND APPARATUS FOR CONTROLLED DELIVERY OF OPIOID AND OTHER MEDICATIONS

(71) Applicant: Somniferum Labs LLC, Las Vegas, NV (US)

(72) Inventor: David Lanzkowsky, Las Vegas, NV (US)

(21) Appl. No.: 15/789,922

(22) Filed: Oct. 20, 2017

Related U.S. Application Data
(60) Provisional application No. 62/411,455, filed on Oct. 21, 2016.

(51) Int. Cl. A61M 11/04; A61M 15/00; A61M 16/22; A61K 31/485; A61K 31/7048

(52) U.S. Cl. CPC A61M 11/042; A61M 15/0021; A61M 16/22; A61B 5/1172; A61K 31/485; A61K 31/7048; A61M 15/009

(57) ABSTRACT

A method, system and apparatus for administering various medicaments including those for treating pain and substance dependency are disclosed. The apparatus is a unit for heat activation of a morphine opiate liquid concentrate mixed with a carrier substance to produce inhaled gas. The method includes inhaling the heat activated gaseous vapor concentrate for pain relief, to treat substance dependency or administration of other medicaments. The system includes a heat vaporization unit with security, control and communication capability to provide effective patient care and ensure safety.

### CROSS-REFERENCE TO RELATED APPLICATION(S)

This application claims priority under 35 USC 119 (e) to U.S. Provisional Patent Application No. 62/411,455, filed Oct. 21, 2016 and titled “Method, Substance, System and Apparatus for Treating Pain and Addiction,” the disclosure of which is hereby incorporated herein by reference in its entirety. This application hereby incorporates by reference in its entirety and for all purposes the disclosure of U.S. patent application Ser. No. ______ filed on even date herewith, naming the same inventor, and entitled “Compositions, Methods and Kits for the Safe Inhaled Delivery of Targeted Opioids for the Treatment of Pain and Addiction.”

### FIELD

The described embodiments relate generally to a medical device, system and method for controlled inhalation of a therapeutic substance. More particularly, the present embodiments relate to the treatment of opioid substance dependency and pain management. In still greater particularity, the embodiments utilize a novel inhalation device to allow controlled administration of opioid compositions. By way of further characterization, but not by way of limitation thereto, the invention relates to inhalation of opioid compositions dissolved in any carrier substance which has been heated to gasify the substance into an inhalable vapor.

### BACKGROUND

Morphine is the most commonly used analgesic for severe pain. It was isolated in 1805, but information about its metabolism and the importance of its metabolites has only emerged since the late 1960s. Because of its analgesic effects, morphine has been widely abused and has become an addictive substance in many instances. Once ingested or delivered intravenously, morphine is predominantly metabolized by hepatic glucuronidation with the addition of the sugar molecule at the phenolic 3-hydroxyl (morphine-3-glucuronide or M3G/M-3-G) or the alcoholic 6-hydroxyl position on the phenanthrene ring (morphine-6-glucuronide or M6G/M-6-G). M-3-G is the morphine molecule responsible for side effects such as addiction, overdose, constipation, etc. In humans, M-6-G is a major active metabolite of morphine, and as such is the molecule responsible for much of the pain-relieving analgesic effects of morphine and heroin.

Substance abuse, sometimes known as drug abuse, is a habitual use of a drug in which the user consumes the substance in amounts, or with methods, which are harmful to the user or others. Widely differing definitions of drug abuse are used in public health, medical and criminal justice contexts. Opioid use disorder is a medical condition that is characterized by the compulsive use of opioids despite adverse consequences from continued use and the development of a withdrawal syndrome when opioid use stops. It involves both an addiction to, and dependence upon, opioids. Opioids are a class of analgesic compounds used medicinally for effective relief of both acute and chronic pain. Opioids bind with the μ G-protein coupled receptor located on the membrane of cells in the brain. Upon binding, opioids act as an agonist, activating the receptor and relieving pain. In addition, opioids are known for alleviating anxiety, inducing mild sedation, and producing a sense of “well-being.”

Medical diagnosis of substance abuse may be based upon a preoccupation with a desire to obtain and take the drug and persistent drug-seeking behavior. The opioid dependence-withdrawal syndrome involves both psychological dependence and marked physical dependence upon opioid compounds. Tens of millions of people worldwide have high-risk drug use otherwise known as recurrent drug use causing harm to their health, psychological problems, or social problems which puts them at risk of those dangers. Hundreds of thousands of deaths result from misuse with the highest number of deaths occurring from opioid use. In some cases, criminal or anti-social behavior occurs when the person is under the influence of a drug. In addition to possible physical, social, and psychological harm, use of some drugs may also lead to criminal penalties in various jurisdictions. Long term personality changes in individuals may occur as well.

Substances most often associated with abuse include: alcohol, barbiturates, benzodiazepines, cannabis, cocaine, methaqualone, opioids and substituted amphetamines. Other substances such as tobacco may also be overused or abused resulting in detrimental physical consequences to the user. Theories as to an individual's proclivity for substance abuse include a genetic pre-disposition, an activity learned from others or a habit which, if addiction develops, manifests itself as a chronic debilitating disease.

Opioids can induce physical chemical dependency, behavioral dependency and tolerance. Aside from the physical aspects of chemical dependency and tolerance, in a relatively small number of cases opioids have been associated with iatrogenic (physician-induced) addiction. Additionally, opioids are at times abused following standard medical pain management. Rates of abuse or misuse of opioids following prescriptions is high. FDA-approved opioids are considered drugs of high abuse potential, and are listed under the Controlled Substances Act as schedule II. Heroin cannot be prescribed in the US and is listed as a Schedule I drug.

Use of opioids is of heightened concern recently due to the quadrupling of the opioid overdose death rate over the past 15 years. Serious side-effects of opioids, typically administered intravenously (IV) or orally (PO), are respiratory depression, hypotension and death. As a result of the growing opioid epidemic, the United States public health officials have initiated a concerted effort to reduce opioid-related deaths. In order to begin to address this issue, a novel paradigm for prescribing opioids and treating chronic pain is needed. Further, new and innovative approaches are needed to safely treat patients with physical chemical addiction, tolerance and behavioral addiction.

Dosing levels can be quite variable, based on the effect of the opioids within each individual patient. Typically, exact dosing regimens are not listed on prescription labels as a result. For example, Oxycontin® tablets range from 10 mg to 160 mg, yet the “Indications and Usage” section states that “[p]hysicians should individualize treatment in every case.”

Opioid dependence requires long-term treatment and care with the goals of reducing health risks for the consumer, reducing criminal behavior, and improving the long-term physical and psychological condition of the addicted person. Most strategies aim ultimately to reduce drug use and lead to abstinence. In recent years, there has been a significant increase of prescription opioid abuse compared to illegal opiates like heroin. This development has also implications for the prevention, treatment and therapy of opioid dependence. No single treatment works for everyone, so several strategies have been developed including therapy and drugs. Detox programs are rarely a good solution, and patients often relapse after going through them. Additionally, the riskiest time for overdose and death for opioid dependent patients occurs within (90) ninety days of discharge from either incarceration or rehabilitation and detoxification.

Opioid replacement therapy (ORT) (also called opioid substitution therapy or opioid maintenance therapy) involves replacing an illegal opioid, such as heroin, with a longer acting but less euphoric opioid; methadone or buprenorphine are typically used and the drug is taken under medical supervision. The driving principle behind ORT is the program's capacity to facilitate a resumption of stability in the user's life, while the patient experiences reduced symptoms of drug withdrawal and less intense drug cravings; a strong euphoric effect is not experienced as a result of the treatment drug. Some patients maintain complete abstinence from opioids while receiving opioid replacement therapy, and others are able to reduce their use. Clonidine or lofexidine can help treat the symptoms of withdrawal.

ORT has proven to be an effective treatment for improving the health and living condition of people experiencing problematic illegal opiate use or dependence, including mortality reduction and overall societal costs, such as the economic loss from drug-related crime and healthcare expenditure. Opioid Replacement Therapy is endorsed by the World Health Organization, United Nations Office on Drugs and Crime and UNAIDS as being effective at reducing injection, lowering risk for HIV/AIDS, and promoting adherence to antiretroviral therapy.

Methadone maintenance treatment (MMT), a form of opioid replacement therapy, reduces and/or eliminates the use of illegal opiates, the criminality associated with opiate use, and allows patients to improve their health and social productivity. In addition, enrollment in methadone maintenance has the potential to reduce the transmission of infectious diseases associated with opiate injection, such as hepatitis and HIV. The principal effects of methadone maintenance are to relieve narcotic craving, suppress the abstinence syndrome, and block the euphoric effects associated with opiates. Methadone maintenance has been found to be medically safe and non-sedating. The individual is prescribed an amount of methadone which is titrated up until withdrawal symptoms subside, followed by a period of stability, the dose will then be gradually reduced until the individual is either free of the need for methadone or is at a level which allows a switch to a different opiate with an easier withdrawal profile, such as Suboxone. Methadone programs often have poor compliance and polydrug abuse is often found in this subset of patients. Suboxone is expensive, few medical providors are licensed to prescribe the drug and compliance rates remain low while recidivism rates remain high.

While many treatments and substances have been tried to treat substance abuse and alleviate pain, there exists a need for a safe, controllable and simple method and system for doing so.

### SUMMARY

The term “opioid composition”, as used herein, refers to opioids, opioid metabolites or a solution including at least one of, or a combination of any two or more of, morphine, morphine sulfate, 6 mono acetyl morphine, morphine-6 glucuronide, morphine-6 glucuronide bromide, morphine-6 sulfate, morphine-6 acetate (the latter four collectively referred to herein as M-6-G), any other pharmacologically acceptable salt of these opioids, or any synthetic, natural or semi-synthetic opioid. Opioid composition, as used herein, may also include soluents for the purpose of opioid stability or therapeutically-acceptable pH, and solvents such as water, polypropylene glycol, glycerin and vegetable glycerin. The term “opioid composition” will also encompass a variety of concentrations as required to optimize the administration of the prescribed dose.

In humans, M-6-G is a major active metabolite of morphine, and as such is the molecule responsible for much of the pain-relieving analgesic effects of morphine and 6 monoacetylmorphine. As used herein M-6-G also includes M-6-PO4,M-6-SO₄,M-6-Glucuronide Bromide, M-6-Glucuronide Acetate and any other M-6-compound. In an embodiment, a morphine/opium substance (targeted opiate) is heat activated and inhaled by a patient to treat pain and addiction. Morphine-6-glucuronide is a preferred substance in one embodiment.

M-6-G may be contained in a tamper proof container, such as a secure ampoule. In an embodiment, the tamper proof container may be double-walled glass, plastic, or metal container or combination thereof with the M-6-G in the inner container and an opioid antagonist such as naloxone, naltrexone, nalmefene, nalorphine, or samidorphan contained between the walls to render the M-6-G unusable or ineffective if mixed with the M-6-G.

The container must be installed and/or removed from the inhalation device by a registered pharmacist or other authorized individual. A key or code may be required to install the container. If an unauthorized attempt is made to remove, install or otherwise access the M-6-G in the container (i.e. by breaking the container), the inner wall breaks first and the counter acting drug within the walls neutralizes the M-6-G or makes it otherwise unusable. Thus this tamper proof container prevents users from accessing the containers to access the M-6-G to inject or otherwise abuse it.

In some embodiments, other substances may be employed in the container to treat other types of substance abuse such as alcohol or tobacco addiction. For example, a nicotine based substance could be used in place of M-6-G to help a person who is addicted to cigarettes and wants to quit smoking. In another embodiment, an ampoule filled with naltrexone solution may be used to treat alcohol dependency. Other medicaments that may be advantageously utilized with the disclosed embodiments include anti-hypertensives, anti-arrythmics, antianginal drugs, anticonvulsants, anxiolytics, antidepressants, antipsychotics, sedatives, hypnotics, opioids, NSAIDS, muscle relaxants, movement disorder drugs, antibiotics, bronchodilators, diabetes medications, thyroid medications, and antibiotics for pulmonary infections.

Opioid compositions may be administered with a heat-activated drug inhalation device. The inhalation or vaporizing device may be used in conjunction with the tamper proof container to dose and otherwise regulate the administration of the substance. The inhalation device treatment is used in place of methadone treatment or other drugs currently being used to treat heroin addiction or for pain relief. In some embodiments the drug or substance may be inhaled and heating or thermal vaporization may not be utilized.

A controller may be included in the device to monitor and regulate the timing and amount of M-6-G dosage. The controller or chip could also be used in the inhalation device to help smokers quit by administering controlled dosing to wean those individuals off of nicotine. The embodiments disclosed herein may also be used to treat alcohol addiction. Similarly, the device could be used to wean patients on Chronic Opioid Maintence Therapy (COMT) off opioids.

One embodiment includes an M-6-G composition that is delivered to the patient by vaporization and subsequent inhalation. The M-6-G composition may be fully solubilized in propylene glycol or other carrier/excipient and may be administered at temperatures from less than about 0° C. up to 600° C. or more in some embodiments, depending upon user preference and safety. Some embodiments heat to about 50° C.-250° C., or 90° C.-110° C. In particular, the lower temperature will be based on the lowest temperature for which the opioid composition changes phase to a gas and on the highest temperature for which minimal decomposition of the opioid(s) occurs and the optimal safety profile of the heat activated opioid composition is assured.

The composition can include one or more therapeutically active doses of the M-6-G, or any other known opioid or other composition. For purposes herein, vaporized refers to a heated vapor state of the targeted opioid. In one aspect of the above composition, the therapeutically active dose of targeted opiate composition may be from 0.01-1000 mg and more typically 0.01-100 mg., while alternate embodiments may dose in the range of 0.01-50 mg or 0.02 to 15 mg., for administration by vaporization every 3-4 hours, or for any time frame and with any breakthrough dosing as determined by the medical provider here-after known as the “prescriber”.

In addition to a tamper proof insert and dosing control, the inhalation technology in the present disclosed embodiments include secure access to the vaporization device using fingerprint, retinal, password, or other security or identification measures. Technologies, including biometric identification (including but not limited to fingerprint retina, or voice), may also be utilized to regulate and limit dose administration.

The controls to limit and regulate dose administration may optionally be adjusted remotely by the health care practitioner via wired or wireless technology (WiFi or Bluetooth) or at the time of the patient's healthcare provider appointment. The medical care professional or “prescriber” can regulate the time interval between dosing, a breakthrough dosing protocol between regular dosing intervals, as needed for breakthrough pain. The prescriber can adjust the temperature of the device, the number of inhalations, the time between inhalations, the time between dosing and the breakthrough time. The prescriber can program the total number of inhalations per day.

The basis for the adjustment of the medication is unique. Most prescribing providers currently use one or two metrics, either the VAS (Visual Analogue Scale) or the NRS (Numeric Rating Score) to adjust the dose of opioids. This often times results in either under treated or overly sedated patients. By utilizing PROMS (Patient Reported Outcome Measures), the device will track four to five different parameters that highly correlate with the patient's quality of life to adjust the dose. These new tools will allow the providers a unique opportunity to closely monitor and prescribe the best available dose to chronic pain patients which has been previously unavailable in any other drug delivery system

Another security feature that may be used in place of, or in conjunction with, the aforementioned control feature is password or key requirements to activate the heat-activated drug inhalation device. Other security or identification measures can be used alternatively or in conjunction with one or more of the aforementioned security features and pulse oximetry may be incorporated into some embodiments of the heat-activated drug inhalation device.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 is a flow chart illustrating one method for treating pain/opioid dependency;

FIG. 2 shows a perspective view of a heat activated inhalation device;

FIG. 3 shows an exploded view of the heat activated inhalation device;

FIG. 4 shows a side sectional view of a tamper resistant container;

FIG. 5 is a schematic diagram of the operation of the heat activated inhalation device;

FIG. 6 is a side sectional view of the device mouthpiece;

FIG. 7 is a functional block diagram of one embodiment of an inhalation device;

FIG. 8 is a perspective view of an alternate inhalation apparatus;

FIG. 9 illustrates an embodiment including devices for making various measurements associated with the patient's medical status;

FIG. 10A is a top perspective view of an alternate embodiment of a heat activated inhalation device;

FIG. 10B is a side perspective view of an alternate embodiment of a heat activated inhalation device;

FIG. 10C is a front perspective view of an alternate embodiment of a heat activated inhalation device;

FIG. 11A is a top perspective view of an alternate embodiment of a heat activated inhalation device;

FIG. 11B is a side perspective view of an alternate embodiment of a heat activated inhalation device;

FIG. 11C is a rear perspective view of an alternate embodiment of a heat activated inhalation device;

FIG. 12A is a top perspective view of an alternate embodiment of a heat activated inhalation device;

FIG. 12B is a side perspective view of an alternate embodiment of a heat activated inhalation device;

FIG. 12C is a rear perspective view of an alternate embodiment of a heat activated inhalation device;

FIG. 13A is a top perspective view of an alternate embodiment of a heat activated inhalation device;

FIG. 13B is a side perspective view of an alternate embodiment of a heat activated inhalation device;

FIG. 13C is a rear perspective view of an alternate embodiment of a heat activated inhalation device;

FIG. 14A is an alternate embodiment of an inhalation device illustrating the attachment of an atomization unit to the device; and

FIG. 14B is an alternate embodiment of an inhalation device illustrating the incorporation of an atomization unit in the device.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims. Like reference numerals denote like structure herein.

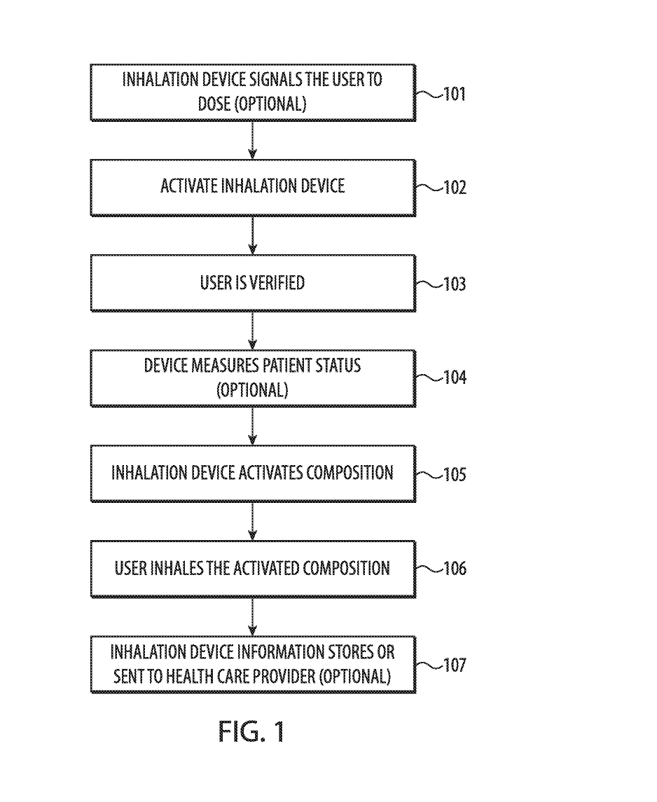

These and other embodiments are discussed below with reference to FIGS. 1-14. However, those skilled in the art will readily appreciate that the detailed description given herein with respect to these figures is for explanatory purposes only and should not be construed as limiting.

Various embodiments disclosed herein are directed toward addressing one or more of the problems discussed above, while prioritizing the patient's health, safety, choice of treatment, reduced adverse effects, and general best interests. An optimal treatment plan will have the added benefits of improvements in social and legal issues for the patient.

A genetic basis for the efficacy of opioids in the treatment of pain has been demonstrated for a number of specific variations. However, the evidence for clinical differences in opioid effects is ambiguous. The pharmacogenomics of the opioid receptors and their endogenous ligands have been the subject of intensive activity in various studies. These studies test broadly for a number of phenotypes, including opioid dependence, cocaine dependence, alcohol dependence, methamphetamine dependence/psychosis, response to naltrexone treatment, personality traits, and others. Major and minor variants have been reported for every receptor and ligand coding gene in both coding sequences, as well as regulatory regions. Newer approaches shift away from analysis of specific genes and regions, and are based on an unbiased screen of genes across the entire genome, which have no apparent relationship to the phenotype in question. These studies yield a number of implicated genes, although many of them code for seemingly unrelated proteins in processes such as cell adhesion, transcriptional regulation, cell structure determination, and RNA, DNA, and protein handling/modifying.

While over 100 variants have been identified for the opioid mu-receptor, the most studied mu-receptor variant is the non-synonymous 118A>G variant, which results in functional changes to the receptor, including lower binding site availability, reduced mRNA levels, altered signal transduction, and increased affinity for beta-endorphin. In theory, all of these functional changes would reduce the impact of exogenous opioids, requiring a higher dose to achieve the same therapeutic effect. There may thus be a potential for a greater addictive capacity in these individuals who require higher dosages to achieve pain control. However, evidence linking the 118A>G variant to opioid dependence is mixed, with associations shown in a number of study groups, but negative results in other groups. One explanation for the mixed results is the possibility of other variants which are in linkage disequilibrium with the 118A>G variant and thus contribute to different haplotype patterns that more specifically associate with opioid dependence.

In one embodiment, users in need of treatment for opioid substance dependency may be initially screened for the 118A>G variant on the mu-receptor. Identified users having the 118A>G variant are at a higher risk of opioid dependence and are therefore at an increased need for the methods, compositions and devices of the present disclosure. Such users are treated with the vaporized morphine-6 glucuronide compositions described herein using the inhalation device as described herein. Such users, having the variant mu-receptor, are at an increased likelihood of overcoming their dependency through the inhalation of the vaporized morphine-6 glucuronide as compared to conventional opioid use (morphine, for example), and administered by conventional (oral or IV, for example) methods. In another embodiment, users in need of chronic pain relief, for example cancer patients, are screened for the 118A>G mu-receptor variant, as these patients are also at increased need of the methods, compositions and devices of the present disclosure. Patients with chronic pain, identified with the mu-receptor variant, may be treated using vaporized morphine-6 glucuronide, as compared to the less predictable conventional opioids and opioid administration. These variant mu-receptor users would gain more consistent pain relief and safe dosing as compared to users that don't carry the variant trait.

The active ingredients in some disclosed embodiments include morphine, morphine sulfate (both FDA-approved analgesics), M-6-G, (M-6-G also includes M-6-PO4,M-6-SO₄,M-6-BR, M-6-acetate or any other M-6 compound) or any other salt forms of the aforementioned substances. Further, morphine or morphine oil concentrate including existing morphine concentrates may be administered with the heat-activated drug inhalation device. In humans, M-6-G is a major active metabolite of morphine, and as such is the molecule responsible for much of the pain-relieving analgesic effects of morphine and 6 monoacetyl morphine.

In one embodiment, the heat-activated drug inhalation medical device is used in conjunction with the tamper-resistant container to administer the opioid composition according to the directions of the prescribing health care provider. The novel heat-activated drug inhalation medical device is used as an alternative or adjunct treatment for opioid dependency to currently recommended treatment options. The device can be used to systematically reduce the opioid dose to taper-off an individual from an opioid drug, in an effort to reduce the adverse effects of opioid withdrawal. For the indication of opioid dependency, a controller (computer chip and software) may be included in the heat-activated drug inhalation medical device to monitor and regulate the timing and amount of opioid administered. The controller could also be used for smoking cessation in the drug inhalation medical device or other drug inhalant medical device which may or may not include heating.

An embodiment uses heat activation of an opioid composition in a liquid state to a gaseous state. Heat activation refers to the physical change of a compound or mixture from liquid (or solid) to gas by applying heat to an appropriate temperature. According to basic thermodynamic properties of mixtures, the temperature required for heat activation will be higher for the mixture than the solvent alone, yet lower than the solute (opioid) because a mixture with a non-volatile compound will lower the vapor pressure of the solvent. The gaseous particles produced by the device will retain a consistent concentration of the active ingredient. The temperature used by the device will prevent the active ingredient (opioid[s]) from decomposition or other chemical alteration, ensuring a safe and consistent dose. Because M-6-Gis a small miscible molecule it completely dissolves in a mixture of VG/PG (Vegetable Glycerin/Propylene Glycol) any mixture or any ratio of VG/PG. The optimum temperature will be the optimal vaporizing temperature that is dependent on the ratio of VG/PG mixture and is not dependent on the solvent.

One embodiment includes an opioid composition that is delivered to the patient by inhalation of the heat-activated opioid composition from the drug inhalation medical device. The opioid composition may be heated to between about 40-180° C. or may be administered at any temperature from above or below freezing up to 600° C. or more depending upon the physical properties of the opioid composition. In particular, the lower temperature will be based on the lowest temperature for which the opioid composition changes phase to a gas and on the highest temperature for which minimal decomposition of the opioid(s) occurs and the optimal safety profile of the heat activated opioid composition is assured.

Optional ingredients for the opioid compositions include other naturally occurring alkaloids like caffeine, chocolate and even nicotine for improved efficacy with certain indications. Additionally naturally occurring flavorings that are Generally Regarded as Safe (GRAS) eg; rose-oil, vanilla, peach etc., may be added. In one embodiment, use of inhaled M-6-G avoids the presence of the other major morphine metabolite, morphine-3 glucuronide, a non-analgesic derivative of morphine. Morphine-3-glucuronide may elicit pain-related side effects including twitching/jerking and may impede M-6-G activity through competitive receptor binding.

M-6-G is one of the 2 primary metabolites (M-6-G and morphine-3 glucuronide) of morphine, converted via the liver. M-6-G has low plasma protein binding allowing for a high availability. Morphine-3-glucuronide is generated in an amount 5 times as much as M-6-G from a given amount of morphine. Although the passage of M-6-G is lower across the blood brain barrier than morphine, plasma concentrations of M-6-G may be approximately 4-fold higher than morphine. Further, M-6-G has a similar affinity to the μ receptor found on the surface of cells in the brain as morphine and morphine-3-glucuronide. The potency, however, of M-6-G is higher by as much as 4-fold than morphine. Without being limited by a particular substance, a heated opioid composition in a gaseous state containing M-6-G, as delivered via the cardio-pulmonary tract, will elicit a more effective analgesic response than morphine. Further, M-6-G does not have as high of an affinity of binding with μ receptors in the gastrointestinal tract as does morphine, resulting in less constipation when doses of M-6-G and morphine are equivalent. Some clinical studies that have investigated M-6-G suggest a better side-effect profile than morphine, including less respiratory depression and nausea. Other side effects which may be reduced with M-6-G compared to other opioids include tolerance and itching. M-6-G is not reported to cause withdrawal symptoms because physical dependence and withdrawal are mediated by μ-2 receptor, not readily bound with M-6-G. M-6-G allergies are unlikely, as it is a naturally occurring endogenous opiate.

While Morphine-6-glucuronide is one preferred M-6-G, it should be expressly understood that, as stated above, other suitable M-6-G substances such as morphine-6-G-BR bromide (hereinafter M-6-GBR), morphine-6 sulfate, and morphine 6 acetate have been found to be stable in alternate embodiments. Another suitable compound is 6 mono acetyl morphine (hereinafter 6MAM) or heroin. Without being limited by a particular substance, it is believed that heated M-6-G vapor, as delivered via the cardio-pulmonary tract, is passed through the Blood Brain Barrier (BBB) to μ receptors found in the brain. The M-6-G, once in the brain, spends more time on the receptors as compared to codeine and other like opiates. The vaporized M-6-G does not interact with μ receptors in the gastrointestinal tract, and does not adversely affect CO₂ levels in the user. The M-6-G also avoids side effects present in most opium and opium derivatives. The lack of thebaine in M-6-G provides a significant increase in the M-6-G effectiveness, as thebaine is responsible for a number of the negative side-effects associated with opiates, including the increase in blood CO₂ levels. In addition, use of vaporized M-6-G avoids the presence of morphine-3 glucuronide, the non-analgesic derivative of morphine. Morphine-3 glucuronide presence may modify M-6-G activity, allowing the two metabolites to compete for the same μ receptors or compete for the overall effect of the combined presence of the two drugs.

Typically, opioid drugs such as morphine are administered, intravenously (IV), intramuscularly (IM), orally (PO), rectally (PR), and sublingually (SL). When given orally or by subcutaneous infusion (IV or IM), morphine is metabolized by the body into M-6-G and M-3-G. M-6-G is responsible for the pain relief while Morphine and M-3-G is responsible for many of the deleterious side effects of morphine substances. The currently disclosed embodiments deliver the opioid composition such as an M-6-G mixture to the brain by inhalation of the gaseous opioid composition. One embodiment utilizes this inhalation method which may result in a safer route of administration than the current standard of care methods used.

The primary advantage of pulmonary delivery over PO is that it permits distribution through the body and to the brain prior to first pass hepatic metabolism (which occurs with PO). Avoiding first-pass elimination in the liver retains the drug for use by the rest of the body. Further, the pulmonary route of administration would naturally inhibit an overdose of the opioid. With IV, IM, and PO administration, opioids can result in an overdose before the effects of the drug are realized, whereas, with inhalation of the opioid composition, the patient is limited by the amount of drug delivered in each breath. The sedative effects of the opioid composition effectively prevent the patient from being capable of administering an overdose, as the novel drug inhalation device requires a level of competency. The loss of this level of competency will very likely occur long before an overdose is possible.

M-6-G compositions administered via inhalation can safely treat opioid dependency utilizing the concept of harm reduction. The safety profile of M-6-G is expected to be superior to Nonsteroidal Anti-inflammatory drugs (NSAIDS). Unlike NSAIDS, M-6-G does not produce peptic ulcers, or increase risk for cardiac problems such as stroke. M-6-G inhalation can safely reduce prescription opioid pill dependency because M-6-G has a lower affinity to the μ2 opioid receptor, thereby reducing the risk of respiratory depression. The use of inhaled M-6-G avoids the presence of the other major morphine metabolite, morphine-3 glucuronide, a non-analgesic derivative of morphine.

Following intravenous (IV) morphine administration in humans, (M-3-G) is the major metabolite accounting for approximately 75% of the total area under the concentration-time curve of morphine and its principle metabolites. The next most abundant metabolite, contributing 15% is M-6-G. One specific h.p.l.c (high performance liquid chromatography) method showed that, following IV administration, M-6-G was present in higher concentrations than morphine from 1 hour onward. Some studies have shown that administration of M-6-G in patients with cancer related pain resulted in a significant majority of patients having useful analgesia lasting between 2 and 24 hours. However, oral or subcutaneous administration of M-6-G includes undesirable side effects as the M-6-G may be metabolized by the patient into morphine (including M-3-G).

M-6-G has from about half to the same potency as morphine. Some studies have demonstrated that IV administration of M-6-G is more potent than morphine with dramatically fewer side-effects, producing virtually no nausea or sedation and significantly less respiratory depression. M-6-G has been shown to possess significant μ1-opioid receptor affinity. Its lesser toxicity may be a result of lower affinity for the μ2 opioid receptor, thought to mediate respiratory depression and nausea. M-6-G has a blood-effect site equilibration half-life of about 4 to 8 hours in a subject, and allows for greater control over the analgesic effect, as compared to morphine and most other opiates.

In one embodiment, a therapeutically active and effective dose or prescribed amount of M-6-G is administered via thermal vaporization inhalation with a heat activated inhalation device. The dose may include M-6-G from about 0.1 to 1000 mg for administration by inhalation every 3-4 hours as loaded in the vaporizer device. In some embodiments, the timing and volume of a dose of M-6-G via the inhalation device can be controlled by the controller chip in the inhalation apparatus. In general, a dosing event of opioid composition such as M-6-G should be over a shorter time frame (i.e. 4 inhalation breaths within 2 minutes), but under some circumstances will require a longer time. In some embodiments, one dose of M-6-G may be administered by inhalation over the course about 30 to about 90 seconds.

A method of delivering an opioid composition such as M-6-G for the treatment of opiate abuse, dependency, tolerance and chronic pain in a manner compatible with harm reduction is illustrated in FIG. 1. Various opioid compositions such as M-6-G substances are described in related application entitled “COMPOSITIONS, METHODS AND KITS FOR THE SAFE INHALED DELIVERY OF TARGETED OPIOIDS FOR THE TREATMENT OF PAIN AND ADDICTION” by the same inventor herein and filed on even date herewith, the disclosure of which is incorporated by reference herein for all purposes. Any of those opioid compositions, including all M-6-G substances, may be advantageously utilized in the presently described apparatus, method and system and, where a specific substance such as M-6-G is disclosed, it should be expressly understood that any M-6-G or other substance disclosed herein may be utilized.

A heat activated inhalation device, used in some embodiments herein, may be hand held, disposable and portable. The inhalation device may have a shell, a mouthpiece, an air inlet, an atomizer, one or more opioid composition storage compartments, one or more pumps, a pressure sensor, a heater, a heat sensor, a battery, heat and pressure control, and optionally an on/off regulator tied to a pulse oximeter and/or a biometric security lock such as a finger print scanner. In alternate embodiments, heat activation may or may not be sued depending upon the inhalation temperature, the type of substance to be inhaled, and the inhalation device to be used.

Referring to FIG. 1, in optional operation 101, the device may signal the user that it is time for a prescribed dose. For example, a visual signal such as an LED or other visually recognizable device may alert the user that it is time for a dose. In other embodiments, an audio signal such as an alarm or buzzer may be used to alert the user. In still other embodiments, a haptic alert in the form of a vibration may be used similar to that used in a mobile telephone to alert the user that it is time for the prescribed dose. A timer or clock in the inhalation device may trigger the alert or it may be a wireless signal sent to the device or to the patient's mobile telephone by the patient's healthcare provider. In still other embodiments, there is no signal provided as the patient self-administers the dosing on an as needed or prescribed basis. In other embodiments a flow meter with indicator may alert the user to the amount of time to inhale, the number of inhalations, the length of the inhalation, the pressure generated by the inhalation, and the termination of the inhalation process.

Either as a result of a signal from the device or upon the user's own initiative, the user may activate the device in operation 102. Activation may take the form of an on/off switch on the device or it may be done remotely by a healthcare provider or electromagnetically through a wireless device. The device may also be automatically activated by the signal in operation 101. Activation may occur in operation 101 or may require a combination of operations 101-104 to fully activate the device. In an embodiment where the user's healthcare provider monitors the device, the device may receive a wireless signal after the healthcare provider has authorized a dose. This wireless signal may serve as the signal in step 101, the activation signal in step 102 and/or the user verification in step 103.

Once the device is activated the user's identity is verified at operation 103. In one embodiment, operations 102 and 103 may be combined such that verification of the user automatically activates the device. Verification of the user may be accomplished by user identification through fingerprint scan, retinal scan, password input, or other security measures on the heat activated inhalation device. In some embodiments the user may need to enter a combination lock unique to the intended user.

In one embodiment, in optional operation 104, various sensors associated with the device may measure the user's medical status. For example, blood oxygen saturation is measured through pulse oximetry technology. A chip within the device may calculate the percent saturation and the device may pass or fail this level of activation according to pre-specified limits stored in the device. Other patient biometrics may be measured as will be discussed herein. In one embodiment, operation steps 103 and 104 or operations 102, 103 and 104 may be combined.

As stated above, the inhalation method and apparatus embodiments disclosed herein may be utilized with various opioids and other substances. Referring to Table 1 below, depending upon the medication to be administered, various patient biometric measurements listed in table 1 may be indicated and the device will perform those measurements. For example, for opioids, administered by the inhalation device and method disclosed herein, sensors such as pulse oximeter, oximeter wave form analysis, heart rate and respiratory rate, spirometry, Etco2, apnea monitor, blood pressure, glucometer, accelerometer, EEG sensor, EMG sensor, EKG sensor, temperature measurement, and GSR monitor may be employed to ensure patient safety.

Table 1 below illustrates monitoring systems which are tailored to the patient's need based upon the patient's current health status and condition to be treated. The chart illustrates that a customizable device can be determined based upon the patient's needs. A computerized algorithm may be used to determine the patient's required monitoring. The chart then determines exactly what monitoring devices are required for that particular patient's disease states. Some patients may have multiple disease states requiring multiple monitors. These monitors can be incorporated into the drug delivery system embodiments described herein for opioids but may also be used in alternate embodiments to treat additional disease states and using additional medications.

For example, a patient may enter his disease states that may include diabetes, chronic pain, and thyroid disease. Based upon the physiological needs of those disease states a recommendation for monitoring will be put forward by the software and then incorporated into the production of a patient centric drug delivery/monitoring device. In this embodiment, the patient galvanic skin response, and heart rate are indications as the efficacy of their thyroid disease and treatment while the diabetic heart rate, galvanic skin response, blood glucose vindication of his medication efficacy as relates to diabetes. Additionally this patient suffering from chronic pain would need to be monitored for oximetry, spirometry, accelerometer to measure their physical activity, because this patient will be at high risk for sudden death the device in his case would include GPS and automatic EMS notification in the event it detects no movement of the accelerometer over a four-minute or other set critical time period. A decrease in blood pressure, decreased heart rate or any other indicator of vital signs in extremis may also be used to indicate an emergency situation requiring emergency treatment. A signal from the device may be sent to appropriate personnel indicating the need for such treatment.

TABLE 1

| | | | | | | Oximeter | | | | | | | |
|---|---|---|---|---|---|---|---|---|---|---|---|---|---|
| | Inhala- | Password/ | USB/ | PROM/ | | wave | Heart | | Respir- | | | | |
| Drug | tional | Biometric | Blue | EMR | Pulse | Form | Rate | Spiro- | atory | Etco | Apnea | Blood | Gluco- |
| Classes | Device | Protection | Tooth | Link | oximeter | analysis | Enable | metery | Rate | 2 | Monitor | Pressure | meter |
| Anti- | * | * | * | * | * | * | * | | | | * | * | |
| hypertensives | | | | | | | | | | | | | |
| Anti- | * | * | * | * | | * | * | | | | * | * | |
| arrhythmics | | | | | | | | | | | | | |
| Antianginal | * | * | * | * | * | * | * | | | | * | * | |
| Drugs | | | | | | | | | | | | | |
| Anti- | * | * | * | * | | | | | | | | | |
| convulsants | | | | | | | | | | | | | |
| Anxiolytics | * | * | * | * | | | * | | | | | | |
| Anti- | * | * | * | * | | | | | | | | | |
| depressants | | | | | | | | | | | | | |
| Antipsychotics | * | * | * | * | | | * | | | | | | |
| Sedative/ | * | * | * | * | * | * | | | | * | * | | |
| Hypnotics | | | | | | | | | | | | | |
| Opioids | * | * | * | * | * | * | * | | | * | * | | |
| NSAIDS | * | * | * | * | | | | | | | | | |
| Muscle | * | * | * | * | | | | | | | | | |
| Relaxants | | | | | | | | | | | | | |
| Movement | * | * | * | * | | | | | | | | | |
| Disorder | | | | | | | | | | | | | |
| Antibiotics | * | * | * | * | | | | | | | | | |
| Broncho- | * | * | * | * | * | | | * | * | * | * | | |
| dilators | | | | | | | | | | | | | |
| Diabetes | * | * | * | * | | | | | | | | | * |
| Thyroid | * | * | * | * | | | * | | | | | | |
| Medications | | | | | | | | | | | | | |
| Antibiotics | * | * | * | * | * | | | * | * | * | * | | |
| Pulmonary | | | | | | | | | | | | | |
| Infections | | | | | | | | | | | | | |
| | | | | | | | | | | | | Multiple | |
| | | | | | | | Doctor | | | | | Drug | |
| | | | | | | | Con- | EMR | | | | Use | |
| | | | | | | | trolled | com- | Over- | | GPS/ | Interface | Patient |
| | | | | | Temper- | | Feed- | patible | dose | CPT | Emergency | Internet | Compli- |
| Drug | Accelero- | EEG | EMG | | ature | GSR | back | via | Protec- | Code | Services | of | ance |
| Classes | meter | sensor | sensor | EKG | Monitor | Monitor | Loop | API | tion | Enabled | Notification | Things | Data |
| Anti- | * | | | * | | * | * | * | * | * | * | * | * |
| hypertensives | | | | | | | | | | | | | |
| Anti- | * | | | * | | * | * | * | * | * | * | * | * |
| arrhythmics | | | | | | | | | | | | | |
| Antianginal | * | | | * | | * | * | * | * | * | * | * | * |
| Drugs | | | | | | | | | | | | | |
| Anti- | * | * | | * | | * | * | * | * | * | * | * | * |
| convulsants | | | | | | | | | | | | | |
| Anxiolytics | | | | | | * | * | * | * | * | * | * | * |
| Anti- | | | | | | | * | * | * | * | * | * | * |
| depressants | | | | | | | | | | | | | |
| Antipsychotics | * | | | | | * | * | * | * | * | * | * | * |
| Sedative/ | * | | | | | * | * | * | * | * | * | * | * |
| Hypnotics | | | | | | | | | | | | | |
| Opioids | * | | | | | * | * | * | * | * | * | * | * |
| NSAIDS | | | | | | | * | * | * | * | * | * | * |
| Muscle | | | | | | | * | * | * | * | * | * | * |
| Relaxants | | | | | | | | | | | | | |
| Movement | | | | | | | * | * | * | * | * | * | * |
| Disorder | | | | | | | | | | | | | |
| Antibiotics | | | | | | | * | * | * | * | * | * | * |
| Broncho- | | | | | | | * | * | * | * | * | * | * |
| dilators | | | | | | | | | | | | | |
| Diabetes | | | | | | | * | * | * | * | * | * | * |
| Thyroid | | | | | | * | * | * | * | * | * | * | * |
| Medications | | | | | | | | | | | | | |
| Antibiotics | | | | | | | | | | | | | * |
| Pulmonary | | | | | | | | | | | | | |
| Infections | | | | | | | | | | | | | |

Upon full activation of the device, if the patient is self-dosed, or if the comparison allows for activation and administration of an M-6-G dose, or a dose is otherwise authorized, the heating of the opioid composition is initiated in operation 105. The device may signal that the dose is ready, similar to that described in operation 101. In one embodiment, the device vaporizes a placebo for inhalation by the subject. The placebo can be propylene glycol or any VP/PG mixture without M-6-G. In typical cases, the subject is not informed as to whether they are receiving a dose of M-6-G or the placebo. The placebo is particularly useful when the method and system are used to treat various types of addiction (smoking, alcohol etc.).

All naturally occurring alkaloids, including M-6-G, are generally heated before being effective. However, in some embodiments, the substance may be administered at room temperature or even at or below 0° C. Operation 105 may include distribution of the specified dose volume of M-6-G to the heating element for the phase transition to a gas. Preparation may also include combining the M-6-G with another component including a carrier such as propylene glycol and heating the combined substance to a temperature sufficient to produce a gaseous physical state. In some embodiments, the heating element in the heat-activated drug inhalation device contacts the opioid composition directly to cause activation.

In addition to using the device and method disclosed herein, in step 105 the pharmaceutical compositions disclosed herein may be administered by inhalation as solution, suspension, aqueous solution, drops, irrigations, nebulized solution wherein solvent included water and all buffers, dry powder where the dry powder carrier includes lactose and other carriers, single-dose dry powder units, liquid soft mist wherein vehicle included water and other buffers, propellant-based solutions and suspensions wherein propellant included all hydrofluoroalkanes, mucoadhesive solutions, and nasal rinses. The inhalation of such compositions may be made using devices such as pressurized meter dose inhalers, dry powder inhalers, breath-actuated dry powder inhalers soft mist inhalers, jet nebulizers, ultrasonic nebulizers, vibrating mesh nebulizers, nasal spray bottles, intelligent inhalers, neti pots, intranasal mucosal atomization devices with or without syringes, intranasal vapor inhalers, squeeze bottles for oral and intranasal inhalation, gas driven spray atomizers, all types of insufflators, spacers used with metered dose inhalers, thermal vaporization aerosol devices.

In operation 106, the patient may inhale the gaseous opioid composition, including, in some embodiments, multiple inhalations over a period of time. More than one heating/inhalation may be required to fully administer the prescribed dose in some embodiments. The set of heating/inhalation operations will herein be referred to as a “dosing event.” In some embodiments, in optional operation 107, the subject's health care provider or other monitor may receive notice that the patient has administered a dose of the M-6-G and may monitor the usage and review data as needed. The data may also be stored in the device for future reference. Safety or other signals to a user or healthcare provider may include LED lights, sounds, vibration, messaging to a cell phone or other electronic device, and the like.

The inhalation device tracks the number of opioid composition doses such as M-6-G administered over a preset time period (e.g. past 24 hours) and compares that number with the predetermined amount allowed for that subject. That information may be stored in the device or sent to the patient's healthcare provider or other monitoring individual over a wireless device included as part of the inhalation device in operation 107. For example, a comparison of the number of M-6-G dosing events over the past 2 hours compared with the allowed dosage for the same amount of time may be stored or sent to a healthcare provider. In some embodiments, the patient may determine the dosing timing (self-administered dosing). Any number of different comparisons and calculations can be used to determine whether the subject is enabled to receive another dose of opioid composition such as M-6-G. In one embodiment, the therapeutically effective dose of targeted opioid is from about 0.1 mg to 1000 mg for administration by inhalation every 3-4 hours or as determined appropriate by a health care provider.

M-6-G can be heated to a wide range of temperatures and mixed with a high range of naturally occurring solvents or carrier substances rendering it suitable for administration by inhalation. In one embodiment, this vaporization temperature is about 90° C.-110° C., while other embodiments may include temperatures ranging from about 0° C. or lower to about 1000° C. or higher. Other solubility/vaping medium carrier or excipient substances can include any vegetable oil, triethelyne glycol, or propylene glycol at all ranges of temperatures and pressures. Dissolving M-6-G in vegetable, glycerin, propoleyne glycol, triethelyene glycol, glycomorph, dhydroglycomorph, glycooxycodomorph, hydromorphoglyce, heroglyce and heating the combined substance to produce inhalable gas may be advantageously utilized with the presently disclosed system embodiments. Any solvent that is GRAS can be utilized.

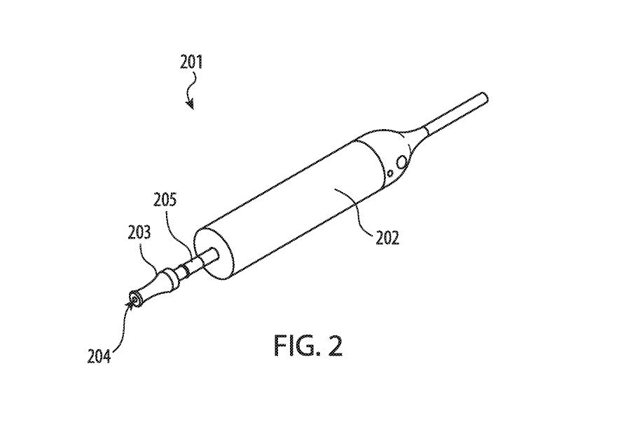

One suitable inhalation device, vaporizing medical device or vaping pen includes a battery connected to a heating coil and set to a pre-determined temperature. The heating coil is connected to a dispensing container with an inhalation port attached. The device may function similarly to a hand held anesthesia machine with much less complexity than other embodiments of the inhalation device. Another embodiment of the inhalation device includes a filter such as a CO₂ scrubber or a soda lime trap to prevent gases exhaled from a user to enter into the atmosphere. This filter prevents bystanders from being exposed to vaporized gaseous opioid composition aerosol from the device. These and other preferred embodiments of a heat activated inhalation apparatus and system for delivery of the opioid or other composition including M-6-G are disclosed below in FIGS. 2-14.

In one embodiment, the administration of M-6-G is metered at precise dosages using the inhalation device. The metering may be accomplished via control of a pump. The inhalation device uses targeted temperatures that will heat activate the opioid composition including M-6-G, but not decompose it or allow derivatives to be formed. In one preferred embodiment herein, M-6-G is combined with propylene glycol (PG) as a solvent, such that the propylene glycol (PG) or vegetable glycerin (VG) or any combination of PG/VG permits a lower activation temperature than solid M-6-G and acts as a carrier of the drug. In some embodiments, the M-6-G is partly solubilized, and in other embodiments the M-6-G is fully solubilized in propylene glycol or any combination of PG/VG.

In some embodiments, the opioid composition including M-6-G can be mixed with caffeine in order to lower the temperature required to convert the opioid composition from liquid to gaseous state (activated). Lowering the activation temperature may be advantageous to users who prefer a lower temperature or are sensitive to higher temperatures. In some embodiments, the substance may be delivered without heating at room temperature or below depending upon the substance and the preference of the user.

In some embodiments, the heating element in the device contacts the M-6-G and propylene glycol solution to cause vaporization, i.e., where the solution changes from a liquid state to a gaseous state. One method for dispensing M-6-G includes heating from between about 90° C. and 260° C. or 30° C.-1000° C. or more in some embodiments in order to form a vapor. While temperatures higher than about 260° C. may be used, patient safety may dictate that lower temperatures be used to prevent burns upon inhalation. Higher temperatures may affect components in the vaporizer and cause contamination from those components to enter the vapor/aerosol. One preferred vaping (inhalational) temperature is about 100° C. In addition to providing solubility, propylene glycol or any combination of PG/VG lowers the vaporization temperature for the opioid composition or other M-6-G.

In a preferred embodiment, the dosing includes a 10% solution of an opioid composition such as M-6-G in glycerol or any combination of PG/VG heated to about 100° C. A patient's pre-existing disease condition such as liver or kidney conditions may dictate adjusting dosage as these are primary elimination routes for opioid substances. In one embodiment, the user is permitted to self-dose up to a certain number (e.g. 10) of doses per day. However, in another embodiment, the dosing user is permitted to administer as needed (PRN). In this embodiment the patient uses it to achieve the desired effect. For example, if the patient desires to decrease anxiety, he may take a dose for depression and then a second dose if he needs pain relief and maybe a third dose if he wants to sleep. Multiple doses of inhaled M-6-G have no detrimental side effects. Addiction and withdrawal occur at the locus Cerueleus located in the pons a site of the brain unaffected by μ1 opioid receptors and therefor unaffected by M6G. Multiple doses of inhaled morphine-6-Glucuronide (M-6-G) will not result in respiratory depression or death for two reasons: first, the user will fall asleep prior to having inhaled sufficient drug quantities needed to affect the breathing center (apneustic centers); and second, M-6-G is a mu-1 agonist that operates above the level of the BBB and does not affect mu-2 receptors (MOR2) largely believed to be responsible for the respiratory depression associated with opioid overdose.

In some animal studies or other models, M-6-G produced potent and long-lasting analgesia, although after morphine administration the metabolite was present in low amounts in small mammals. M-6-G may be effective in relieving breathlessness although it is still uncertain whether such actions are mediated locally or via central mechanisms. In some applications, nebulized M-6-G has been used but, because it is not heated nor mixed with a solvent or carrier to enhance inhalation and delivery to the patient's lungs as in some disclosed embodiments herein, its effectiveness is limited.

In various embodiments, opioid compositions such as M-6-G may be administered by pulmonary delivery via inhalation in the form of inhalable vapors that facilitate the delivery of pharmaceutical preparations that release the active ingredients for a wide range of pharmaceuticals. In various embodiments, vaporized M-6-G such as M-6-G preparations and substances may be used for the treatment of acute and chronic pain such as in place of or in conjunction with prescription synthetic narcotics and pain relief medication.

In various embodiments, the opioid composition may incorporate advanced drug delivery technology, such as liposomes. In this example, the opioid is encapsulated in a lipid layer, then dissolved in a solvent, heat-activated and inhaled. In various embodiments, the opioid composition/inhalation methodology may be combined with other pharmaceutical treatments which have been fully evaluated for safety and adverse drug interactions.

The disclosed vaporized M-6-G embodiments may be used for the treatment of endocrine, musculoskeletal, cardiovascular, cardiopulmonary, respiratory, neurological and psychiatric diseases and disorders, substance use disorders, substance dependence, substance withdrawal, substance addiction, and substance overdose. The disclosed embodiments may be used for the prevention and treatment of disorders and diseases, generated by or acting on the central nervous system. Vaporized M-6-G may significantly lower health care costs by replacing expensive pain or anesthesia treatments.

Referring to FIG. 2, a single-user medication dispensation device is shown that controls or adjusts the volume of opioid composition dispensed by inhaled administration. In one embodiment, an inhalation device 201 has a shell 202, and a mouthpiece 203. A user, not shown, places his or her lips on the mouthpiece and inhales through the mouthpiece. The heat activated M-6-G substance is dispensed through the orifice 204 in the mouthpiece. In some embodiments, mouthpiece 204 may include a filter device 205 such as a CO₂ scrubber or a soda lime trap to prevent gases exhaled from a user to enter into the atmosphere.

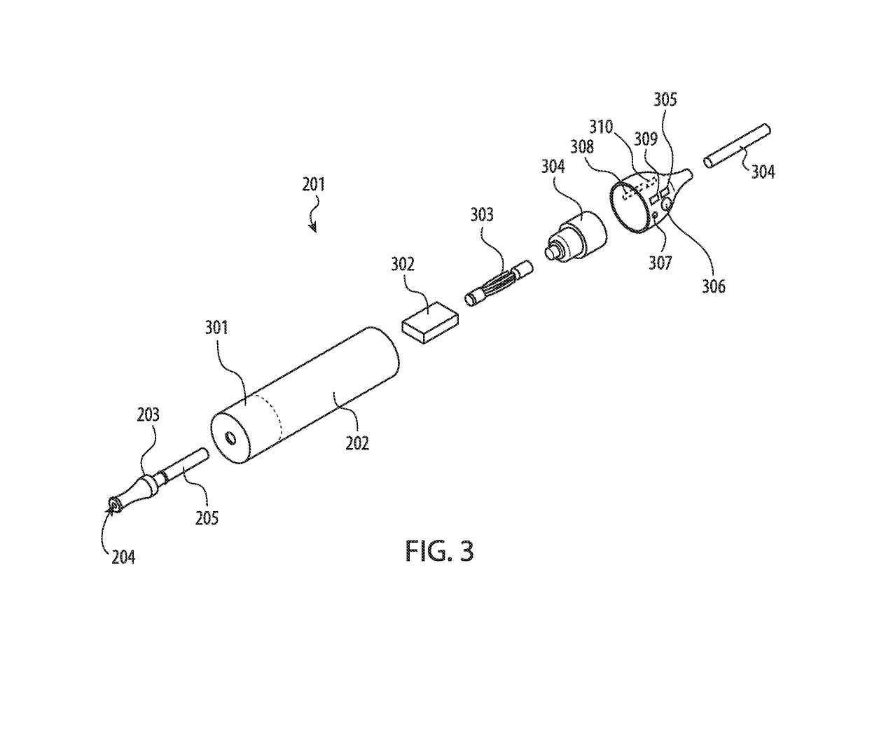

Referring to FIG. 3, the inhalation device 201 is shown in an exploded view. The device includes an atomizer 301, and one or more storage compartments 302 containing an opioid composition such as M-6-G or other substance. In one embodiment, containers 302 may include tamper proof containers as disclosed herein. Device 201 includes a heating element 303, a power supply which may include a battery 304, a wireless transceiver 305 for sending and receiving wireless signals, and a device activation unit 306 which may include an on/off regulator which may optionally include a finger print scanner 309 (which itself may optionally include a pulse oximeter) all as part of an on/off button 306 on device 201. An indicator device 307 which may be a light emitting diode (LED) or other indicating device such as an audible alarm or haptic vibration element is also shown. The device 201 also may include a controller 308. The inhalation device 201 can be made disposable. In some embodiments, it may be a one (or more) use device that is not refillable or it may permit the storage compartment 302 to be refillable or replaceable to permit longer term use of device 201. In some embodiments controlled activation, including physiological monitoring are available via an LCD screen which may include a touch screen.

Controller unit 308 may execute instructions and carry out operations associated with inhalation device 201 as described herein. Using instructions from device memory, controller 308 may regulate the reception and manipulation of input and output data between components of the electronic device. Data transferred to or from the device may be encrypted to comply with HIPAA or other regulations. Controller 308 may be implemented in a computer chip or chips. Various architectures can be used for controller 308 such as microprocessors, application specific integrated circuits (ASIC's) and so forth. Controller 308, together with an operating system may execute computer software code and manipulate data. The operating system may be a well-known system or a special purpose operating system or other system as may be known or used. Control device 308 may include memory capability to store the operating system and user or other data. Control device 308 may also include application software to implement various functions associated with the portable electronic device. For example, an algorithm may be programmed into control device 308 to regulate the timing and amount of dosing for the opioid composition contained within container(s) 302.

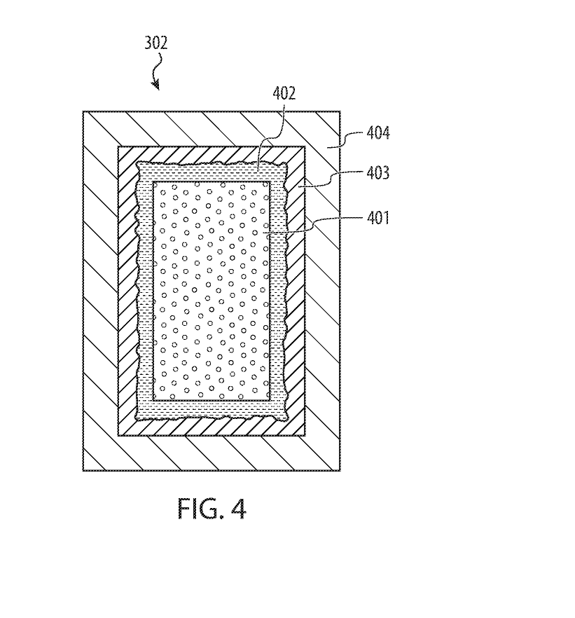

Referring to FIG. 4, tamper resistant container 302 may include the opioid composition substance 401 enclosed in a thin glass or other easily breakable container wall 402. A second outer wall such as a thicker glass wall 404 encloses a substance 403 which may be an antidote type substance such as naloxone in one embodiment. The container or ampoule 302 is constructed such that an attempt to access the M-6-G 401 by a user or other individual through outer wall 404 will result in breaking the inner wall 402 and the mixing of the antidote substance 403 with the M-6-G 401. Thus, attempts by an individual to access the M-6-G for purposes of injection or other disallowed uses will result in either destruction of the M-6-G altogether or rendering it unusable. The personal protection/tamper resistant vial 302 could also be incorporated into existing morphine and or other controlled drug pumps. As described herein, the M-6-G is accessed from the tamper resistant container by inhalation device 201 through a syringe or other permitted method within the device.

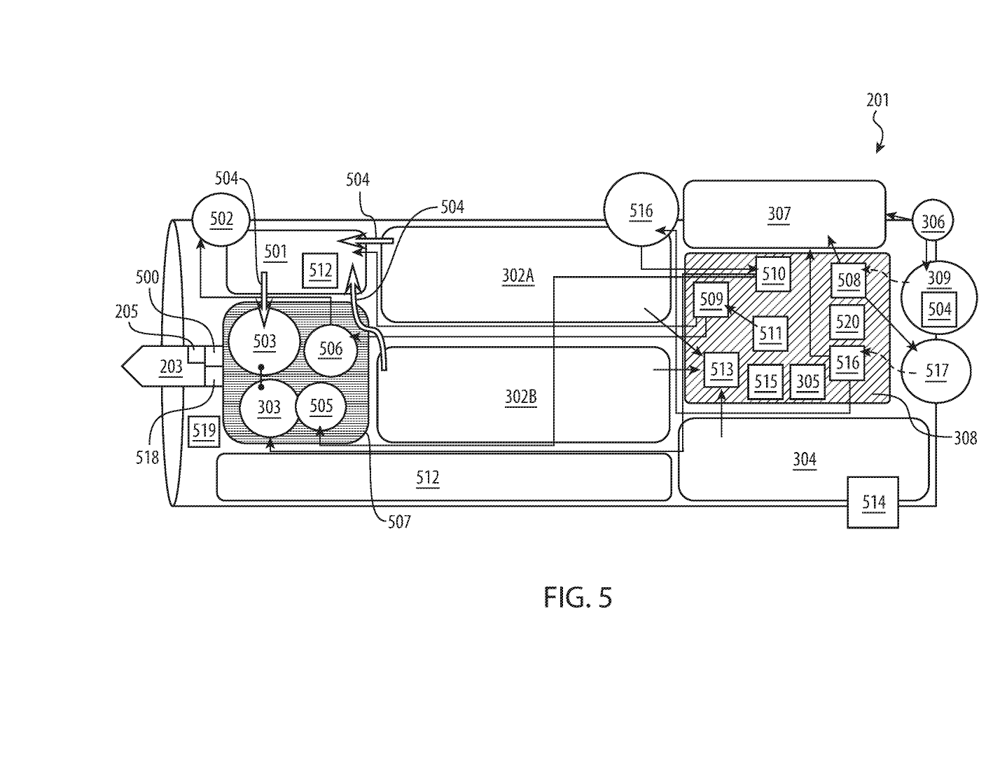

Referring to FIG. 5, a schematic view of the systems, apparatus, and processes associated with device 201 is shown. The opioid composition is administered through a mouthpiece 203. In one embodiment, mouthpiece 203 may include a filter device 205 such as a CO₂ scrubber or soda lime trap to prevent exhaled gases from a user to enter the atmosphere. In another embodiment a flow meter 519 regulates the duration, amount of medication, and/or number of inhalations for optimal dosing.

Referring again to FIG. 5, in one embodiment, two cartridges or ampoules 302A and 302B are shown. In one embodiment, cartridge 302A contains an opioid composition such as M-6-G while cartridge 302B contains propylene glycol or other excipient. In some embodiments the locations of cartridges 302 A and B could be reversed. Alternatively, both cartridges 302A and 302B could contain the same opioid composition or differing concentrations of opioid compositions. One or more pumps 501 access cartridges 302A and 302B as indicated by arrows 504 through syringes or other apparatus 512.

In one embodiment, an active ingredient such as an opioid of M-6-G composition from cartridge 302A and solvent such as propylene glycol or any PG/VG mixtures from cartridge 302B are pumped as indicated by arrow 504 to a reservoir 503 which may be ceramic or other suitable material where the mixture is heated by heater 303. In some embodiments, the substance may be heated to a temperature between about 90° C. to about 260° C. In one preferred embodiment the temperature may be approximately 100° C. Heater 303 may include a pulse width modulation heating element and may include a titanium heating element embedded in a vapor chamber 507.

Vapor chamber 507 may include a heat sensor 505 to monitor the temperature of the heated opioid composition in reservoir 503. A pressure sensor 506 is also included in vapor chamber 507 to monitor the pressure within the chamber. In one embodiment, the pressure monitor may sense a decrease in pressure to indicate that the gaseous substance was delivered to the user.

In some embodiments a syringe with an antidote or other antidote administration device 512 will be housed within or attached to the inhalation device 201 for easy access in the event of an emergency. An indicator such as LED light or other visual, auditory or haptic signal from device 516 may inform the user that the device 201 is ready for a dose administration. A button or switch 518 may be activated by the user to initiate the dose administration.

Referring again to FIG. 5, controller unit 308 may include programmable devices 508 which can store and match biometric or other security data or measure wavelengths of light from the pulse oximeter 517 and thereby calculate oxygen saturation. Programmable devices may include microprocessors, computer chips, clocks 520, CPU's or application specific integrated circuits (ASIC's) powered by battery 304. Controller unit 308 includes a pump control 509 electromagnetically connected to pump 501. In some embodiments, pump control 509 may be used in conjunction with flow meter 519 to control the amount or timing of a dose delivered to the patient. Controller unit 308 includes a heat control device 510 to monitor and regulate the heater 303 and a pressure control device 511 to monitor and regulate the vaporized substance provided to the user through a controlled two-way valve 500 associated with mouthpiece 203. The user inhales the vaporized substance, and then exhales through the mouthpiece/two-way valve. Soda lime trap 205 traps any remaining vapor exhaled to ensure it isn't released into the environment. Signals from heat sensor 505 and pressure sensor 506 may be provided to controller 308 to allow controller to monitor heater 303 and pressure control device 511.

In some embodiments the two-way valve inlet will measure carbon dioxide in the exhaled air through infrared capnography technology 518 as an additional health pre-dose safety component of the device 201. This measurement of carbon dioxide will occur prior to entry into the soda lime trap 205. Carbon dioxide measurements provide a method for early detection of the risk for hypoventilation, hypercapnia, and early indication of opioid overdose. A carbon dioxide measurement above 36 mmHg or other clinically relevant ceiling value would indicate an above-normal reading and not allow a dose, until a safe level has been measured. In some embodiments, a distress signal will be sent out to local medical emergency authorities when an unsafe carbon dioxide measurement has been measured.

One or more valves/syringes/solenoids 512 connected to pump 501 are used to access the opioid composition or other substance to be heated and control the dose delivered to the user by accessing a predetermined amount of substances from cartridges 302A and 302B and providing it to reservoir 503 as shown by arrow 504. Valves/syringes/solenoid 512 may include one or more of: 1) a micro-motor which controls a micro-valve on cartridges 302; 2) a micro-syringe; 3) or a solenoid or rotating valve on cartridges 302. In some embodiments, one or more sensor(s) 513 in controller 308 monitor(s) the amount of the substance in cartridges 302A and 302B as well as the charging level of battery 304. A signal may be provided by sensors 513 to display 307 to indicate these levels and amounts. In one embodiment, cartridges 302 A/B may contain up to 5 ml or more of the substance. In another embodiment a simple screw cartridge is utilized. The screw cartridge is a left handed screw or another unique screw to prevent use in any other device than the intended device.

In one embodiment, battery 304 may be connected to a USB port 514 for re-charging. Port 514 also allows programming and reprogramming of controller 308. The patient connects inhalation device 201 to a computer through a wired connection with USB port 514. Once connected to a computer or patient record, the inhalation device can, in some embodiments, securely connect to the patient portal or upload stored data to the patient's electronic medical record. In some embodiments, in order to charge and use device 201 again the patient may be required to answer specific questions about side effects, functioning, drowsiness and euphoria which the health care provider can review as needed. In some embodiments, a wireless transceiver 305 may be included in device controller 308 to permit communication of information to and from device 201 and to and from other wireless devices such as wireless telephone devices (not shown). The wireless connection may be made directly with the user's electronic medical records or in some instances to other individuals such as family members associated with the user who may be authorized to monitor use. In some embodiments a Global Positioning System (GPS) device 515 may be included in the device to allow the healthcare provider or other authorized individuals to determine the location of the patient for monitoring or in the event of an emergency.

An application for the patient may be available via USB or blue tooth. The patient's portal will be a resource for the patient to access health records, dosage, usage and activities of daily living. The portal can interface with the Health Care Provider's portal for sharing of valuable health information. For example, the PROMIS medical website portal may be utilized for data collection in conjunction with device 201.

Display 307 may also be included in the device to inform the user of various functions and status of the device. For example, display 307 could include LCD or LED lights as part of an indicator unit 516 to indicate: that the device is on; the substance levels in cartridges 302A and/or 302B; that the battery 304 is charged or needs to be charged; that the user is due for a dose of the substance from cartridge 302A; and/or that the user may not access the substance in cartridge 302A either because the most recent dose was administered too recently or the user is denied access for some other reason. For example, access may be denied remotely by the user's health care provider or other authorized person. Display 307 could also include a light or other device 516 to indicate that one or both of cartridges 302A or 302B need to be replaced or refilled because they are either empty or are defective in some way as sensed by sensors 513. Display 307 could also include a dose initiating indicator so that the user is informed that the substance is suitably heated and is ready for inhalation again as indicated by sensors 513. For example, an on/off indicator light 516 may be included in display 307. As discussed above, haptic feedback such as vibrations or audio feed back in the form of audible sounds may be generated as part of indicator device 516 in conjunction with, or in lieu of, display 307.

In order to ensure that each inhalation device is usable only by a single user, device 201 may be protected by a secure access device 309 which may include one or more of the following: a password; a biometric scanner 504 which may include a retinal scanner; and/or a fingerprint scanner. In some embodiments, a pulse oximeter 517 may also be included to sense the oxygen level of a user's blood in conjunction with or in addition to secure access device 309 connected to microprocessors 508 and 502 in controller unit 308. The data from pulse oximeter 517 is supplied to controller 308 and may be provided to the user's healthcare provider through wireless connection 305 or stored for later data transfer. The data from pulse oximeter 517 may also be used by controller 308 in device 201 to control the amount or timing of dose administered to a patient. Data transferred to or from device 201 may be encrypted or otherwise protected to comply with HIPAA or other privacy regulations.

In some embodiments microprocessor or other programmable device 508 may be programmed with an algorithm that is based on the patient's responses to a set of queries presented through display 307 or another user device such as a portable telephone or laptop computer connected through port 514 or wireless connection 305. The user's response to the questions presented could initiate a signal to controller 308 to permit user access to a dose of opioid composition or other substance. The algorithm may adjust the M-6-G dose up or down by, for example, 10% depending upon the responses. The program may automatically adjust the dose to wean individuals off of the opioids. For example, the program may adjust the dose to the lowest dose needed for the patient to perform daily functions. The program may give feedback to the physician and chart out the patients' responses or other data. The program may allow for data mining of actual device use plotted against patient function.

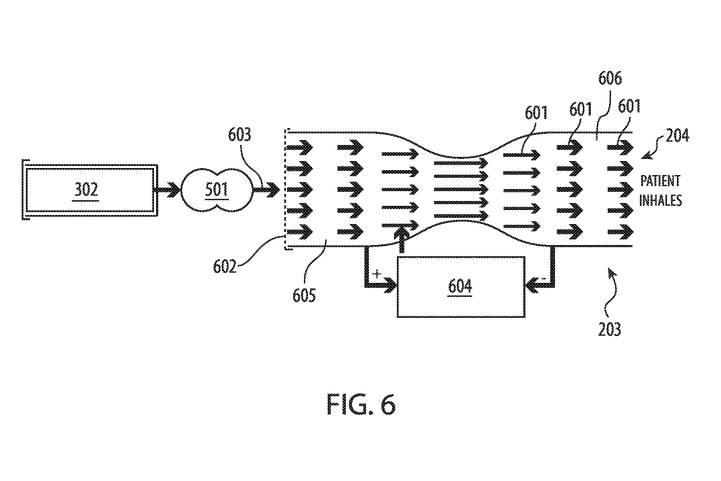

Referring to FIG. 6, mouthpiece 203 is shown in a side functional view. A plurality of mixing vanes 601 may be used to disperse the M-6-G/glycerol mixture from cartridge 302 through pump 501 for inhalation by a user through orifice 204. A cover screen 602 may be employed at one or both ends of mouthpiece 203 to prevent inhalation of unwanted materials. The M-6-G/glycerol mixture is injected into the mouthpiece 203 at injection ports 603 while pressure transducer 604 monitors the pressure differential between chambers 605 and 606 of mouthpiece 203.

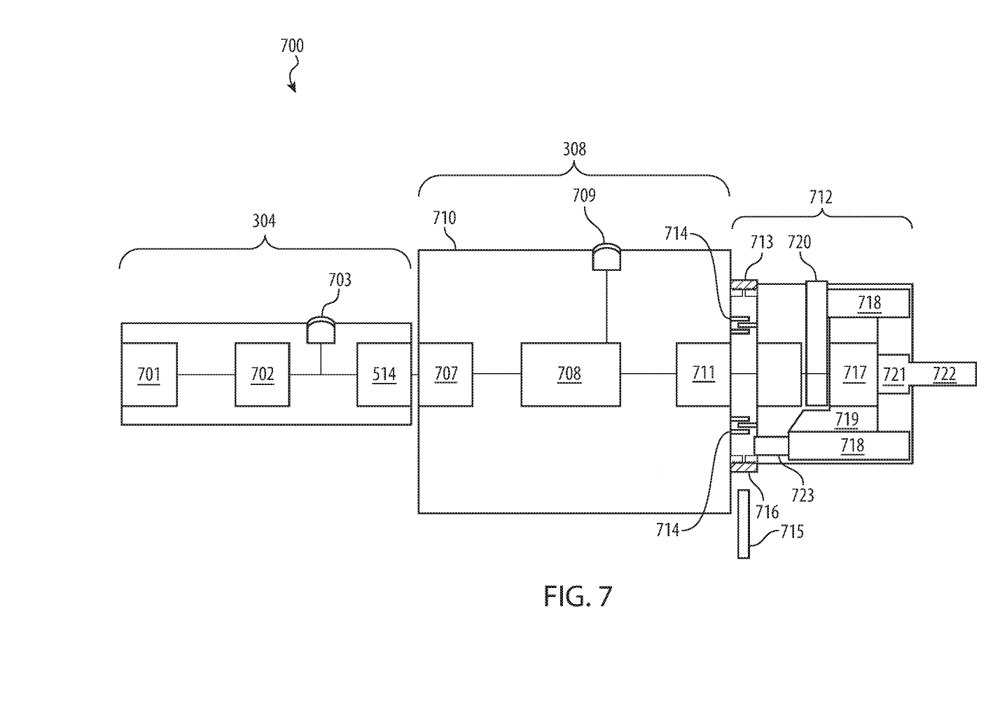

Referring to FIG. 7, a side view functional block diagram of one embodiment of an inhalation system 700 is shown. Power supply 304 may be a commercially available lithium ion cell power pack including charger 701 and battery unit 702. In one embodiment, a pushbutton 703 may be used to energize the power supply. A USB charger 514 may be included with, for example, a standard 2-pin female connector to connect power supply 304 to control unit 308. Control unit 308 may include a 2-pin male connector 707 to mate with female connector in charger 514. Connector 707 is electrically connected to electronic circuitry 708 which may be on a flex circuit or stack of PCBs. An LED indicator 709 may be included to indicate when the device is loaded with M-6-G and ready for inhalation. Control unit 308 may be a metal or plastic tube with connector 707 in one end and a 4-pin connector 711 at the opposite end. While certain types of connectors have been disclosed, the embodiments may include other types of connectors without departing from the scope of the described embodiments.

Referring to FIG. 7, an atomization unit 712 couples to control unit 308 with a ring 713 simultaneously having both left hand and right hand threads. To assemble, atomizer unit 712 plugs into control unit 308 with the ring 713 around the connection, and the user rotates the ring 713 until snug thereby pulling the two parts 308 and 712 together until they mate firmly without rotating them with respect to one another. A 4 pin male connector 714 mates with connector 711. The ring can't be easily removed without a tool 715 because it has a smooth and featureless exterior, except for a uniquely sized or shaped hole 716 to accept the corresponding sized or shaped tool 715. The unit is thus not easily disassembled thereby preventing a child or other unintended user from disassembling it without tool 715.

Access to tool 715 may be limited to authorized users such as medical personnel and pharmacists who are authorized to dispense the M-6-G in the container. Thus, installation and removal/replacement of cartridge reservoirs 302 is limited to these individuals. However, because the M-6-G is desirable by addicted individuals or other unauthorized dealers or individuals, the double walled tamper proof container acts as an additional safeguard to prevent unauthorized access to the contents of the container. In some embodiments, reservoir 302 will contain an RFID tag for inventory control, insuring the proper dose is associated with the proper device. The RFID tag can also help to prevent hoarding or obsolescence of drugs by disabling use after the prescription period ends.

Referring to FIG. 7, atomizer unit 712 includes a heating coil 717 connected to 4-pin connector 714. In one embodiment, heating coil 717 may be a Kelvin-connected coil of 316L SS to support temperature control. In another embodiment, coil 717 may be a pulse width modulated titanium heating coil embedded in the heating chamber. In one embodiment, a reservoir 718 may circularly surround atomizer 712. Reservoir 718 may contain M-6-G and propylene glycol PV/VG, or other carrier substance which has been mixed with the M-6-G. In another embodiment, unit 712 may contain two separate reservoirs 302, one for M-6-G and one for the carrier substance such as propylene glycol. One or more wicks 719 may be used to transport the M-6-G and the carrier substance to the coil 717 where the M-6-G and carrier substance are heated to vaporization temperatures. In other embodiments atomizer unit 712 may be a disposable screw-on, stand-alone, cartridge. An air inlet 720 creates an air path 721 from the side(s) of unit 712 adjacent heater 717 to allow vaporized M-6-G to be inhaled by a user through a mouthpiece 722. Ambient air may cool the vaporized M-6-G to allow a user to safely and comfortably inhale it. A one-way fill port 723 is located inside the locking ring 713 to prevent a child or other unintended user from accessing it. The vaporizer can be loaded with one or more therapeutically effective doses of M-6-G, where one dose at a time is dispensed from a storage chamber or cartridge for vaporization in the vaporizer where appropriate. As stated above, while certain types of connectors such as 2 pin and 4 pin connectors are disclosed, the embodiments described herein may include various types of connectors as are known in the art.

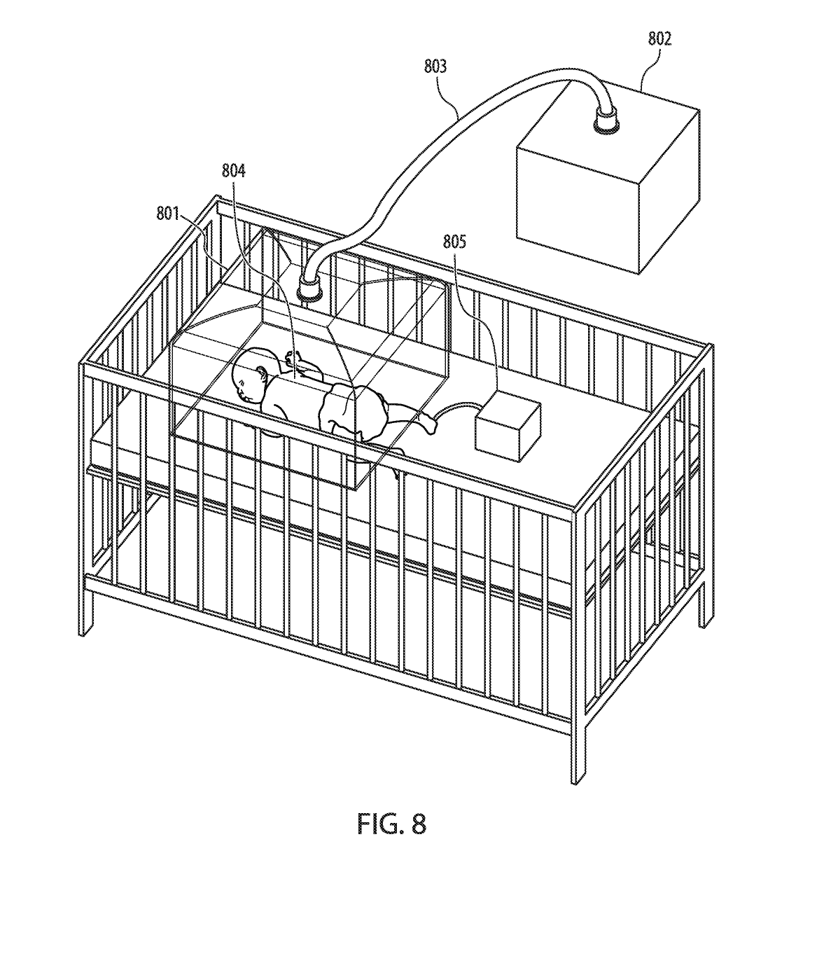

In some embodiments, an apparatus that is not operated by the user including an electronic heat activated drug administration device that emits a drug in a gaseous state for treatment of a variety of medical conditions may be used to administer opioid composition including M-6-G. Every 53 minutes a child is born addicted to opioids in the USA, known as NWS (Neonatal Withdrawal Syndrome). M-6-G has a markedly more favorable profile than methadone. Because of the favorable profile, M6G can safely and quickly wean NWS children easily off the opioid and prevent opioid withdrawal. For example, referring to FIG. 8, in the case of an infant or other patient 804 in a hospital or in other controlled patient situations where patient 804 is not able to inhale directly from a device, a tent 801, in conjunction with a nebulizer or other inhalation device 802, may be used to administer the M-6-G. In some embodiments, inhalation unit 802 may be included inside tent 801 and in some embodiments unit 802 is located outside tent 802 and connected to tent 802 by a hose 803. In this manner, inhalation of the opioid composition or other substance can be used to treat babies born with opioid dependence (via maternal opioid addiction or otherwise) via tent 801 (such as a neonatal tent) which is much safer than conventional treatments. For an adult patient 804, an oxygen tent or other similarly enclosed area may be employed. Inhalation unit 802 may be similar to the embodiments described herein but, rather than a user inhaling directly from the unit, the gaseous opioid composition is administered to the user through hose 803 connected to, or from unit 802 located inside, enclosed space or tent 801.

Tent 801 may consist of a canopy placed over the head and shoulders, or over the entire body of a patient 804 to provide the gaseous opioid composition substance. Some tents cover only a part of the face. This form of administration is often prescribed in conditions where an infant or other patient has difficulty in breathing or where inhaling from an inhalation device is otherwise impracticable. Tent 801 can be used in either a hospital setting or inside or outside of a health-care facility, or for home use, and can be employed for short- or long-term therapy. Typically, tent 801 is made of transparent impermeable material such as plastic.

The tent can envelop the patient's bed, chair, or other reclining area with the end sections held in place by, for example, a mattress to ensure that the tent provides appropriate containment of the gaseous opioid. The enclosure 801 often includes a side opening with a zipper. In some embodiments, medical apparatus for facilitating the inhalation of pharmaceutical preparations or drug delivery systems or other medical apparatus for introducing pharmaceutical preparations into the human body may be used to administer opioid compositions. In some embodiments, a monitoring device 805 is attached to the patient 804 to ensure the patient's safety during dosing. The monitoring device 805 may include one or a combination of two or more of the following: a pulse oximeter, a heart monitor, a CO₂ scrubber or a soda lime trap.

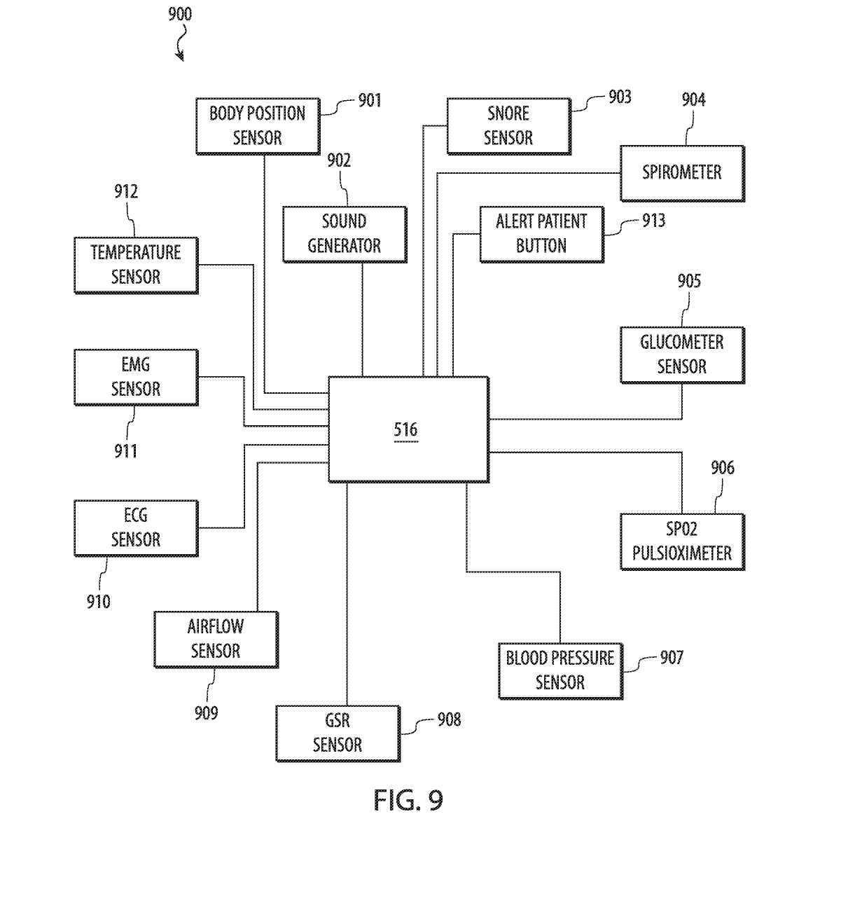

As stated above, in optional operation 104, the user's medical status may be measured by various sensors associated with the device. As shown in Table 1, the types of patient measurements may be indicated by the type of medication to be inhaled. Referring to FIG. 9 various measurements associated with the patient's medical status may be made. In one embodiment, indicator unit 516 and/or monitoring device 805 could include wireless or wired connections to various sensors. For example, in some embodiments, one or more of a body position sensor 901, a sound generator 902, a snore or apnea sensor 903 and a spirometer 904 could be associated with the indicator unit 516 or monitoring device 805. One or more of a glucometer sensor 905, pulse oximeter 906, blood pressure sensor 907, galvonic skin response unit 908, airflow sensor 909, electrocardiogram sensor 910, electromyogram sensor 911 and temperature sensor 912 may also be included to monitor the patient's medical status. In some embodiments, a patent alert button 913 may also be included to allow the patient to access a medical care professional in the event he or she deems it necessary or in case of emergency.

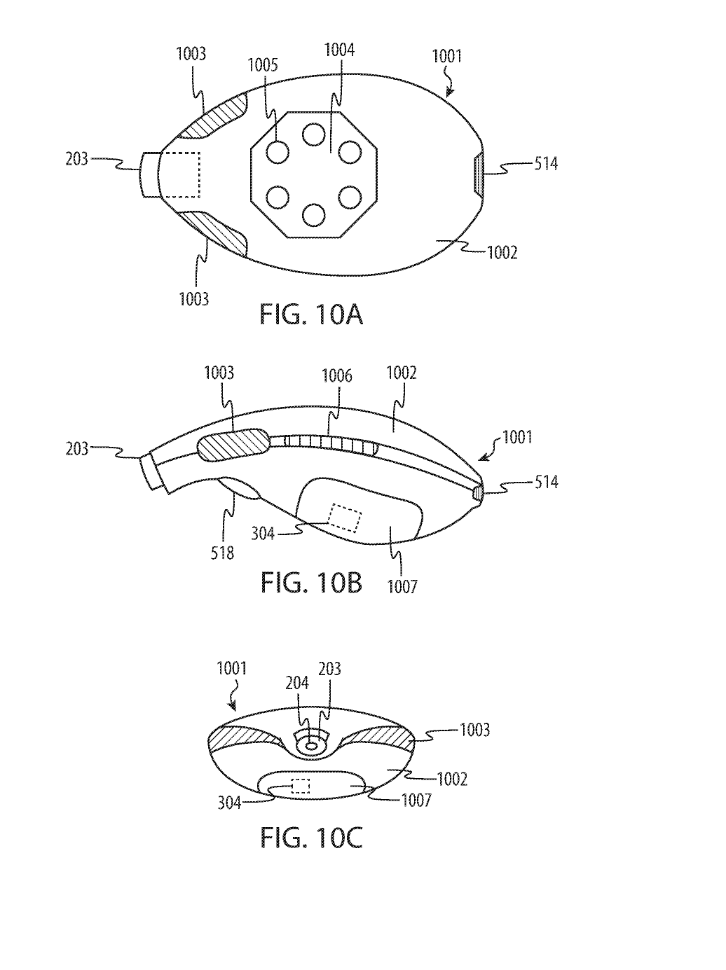

Referring to FIG. 10A, a top perspective view of an alternate inhalation device 1001 is shown. Device 1001 includes an alternate external design but may incorporate some or all of the features and functions described herein. Device 1001 includes a housing 1002 which includes an integrated mouthpiece 203 as previously described. Device 1001 may also include one or more side switches 1003 which may be used for activation or selection of options in the device. Device 1002 may also include USB port 514 as previously described. A keypad 1004 may also include one or more touch keys 1005 for selecting various options on device 1002. Keypad 1004 may also include indicator 307 as previously described to inform the user of various functions and status of the device.

Referring to FIG. 10B, a side view of device 1001 is shown which illustrates mouth piece 203. Device 1001 may also include activation button 306 and a battery level indicator 1006. Alternatively, indicator 1006 could be incorporated into display 307 as previously described. Device 1001 may also include sensors and additional apparatus and devices shown and described herein. An activation button 518 as previously described may also be incorporated in lieu of or in addition to side switches 1003. A rechargeable battery 304 may be included inside the device housing with a battery compartment cover 1007 concealing the battery. The battery 304 may be replaceable by removing cover 1007 or may be recharged through USB port 514 as previously described.

Referring to FIG. 10C, a front view of device 1001 is shown illustrating mouthpiece 203 including orifice 204, side switches 1003 and battery compartment cover 1007 all as shown placed on or in housing 1002.

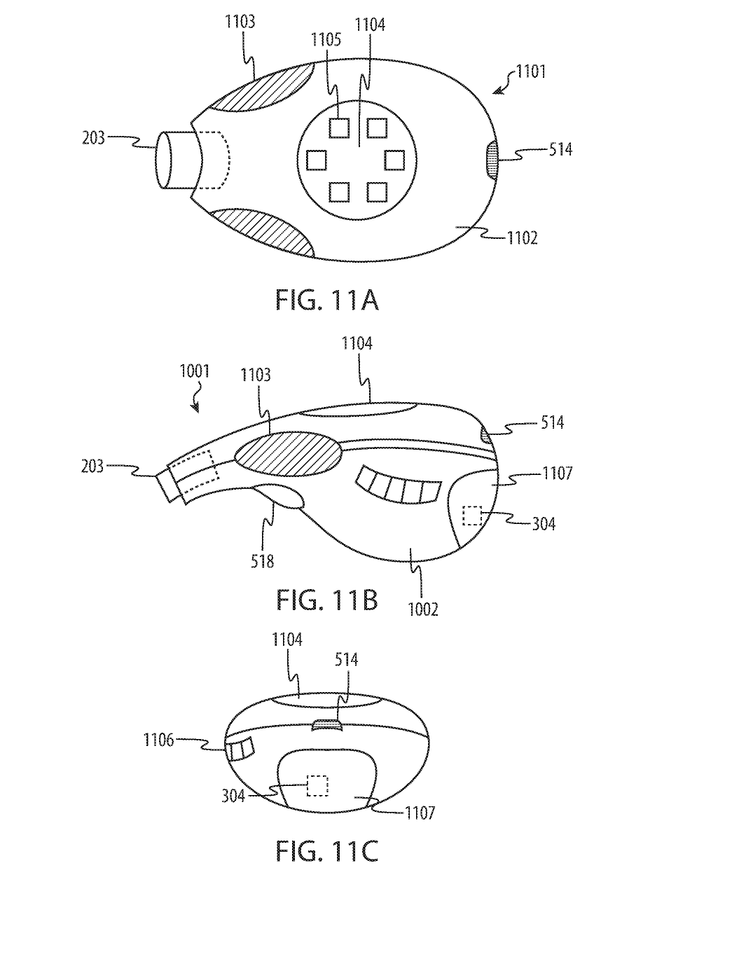

Referring to FIG. 11A a top perspective view of an alternate inhalation device 1001 is shown. As with the embodiment shown in FIG. 10, device 1101 includes an alternate external design but may incorporate some or all of the features and functions described herein. Device 1101 includes a housing 1102 which includes an integrated mouthpiece 203 as previously described. Device 1101 may also include one or more side switches 1103 which may be used for activation or selection of options in the device. Device 1102 may also include USB port 514 as previously described. A keypad 1104 may also include one or more touch keys 1105 for selecting various options on device 1102. Keypad 1104 may also include indicator 307 as previously described to inform the user of various functions and status of the device.

Referring to FIG. 11B, a side view of an alternate embodiment of a device 1101 is shown which includes mouth piece 203. Device 1101 may also include activation button 306 and alternate placement of an indicator 1106 and a battery compartment 1107. Alternatively indicator 1106 could be incorporated into display 307 as previously described. Device 1101 may also include sensors and additional apparatus and devices shown and described herein. An activation button 518 as previously described may also be incorporated in lieu of or in addition to side switches 1103. A rechargeable battery 304 may be included inside the device housing with a battery compartment cover 1107 concealing the battery. The battery 304 may be replaceable by removing cover 1107 or may be recharged through USB port 514 as previously described.

Referring to FIG. 11C, a rear view of device 1101 is shown illustrating USB port 514, indicator 1106, and battery compartment cover 1107 all as shown placed on or in housing 1002.

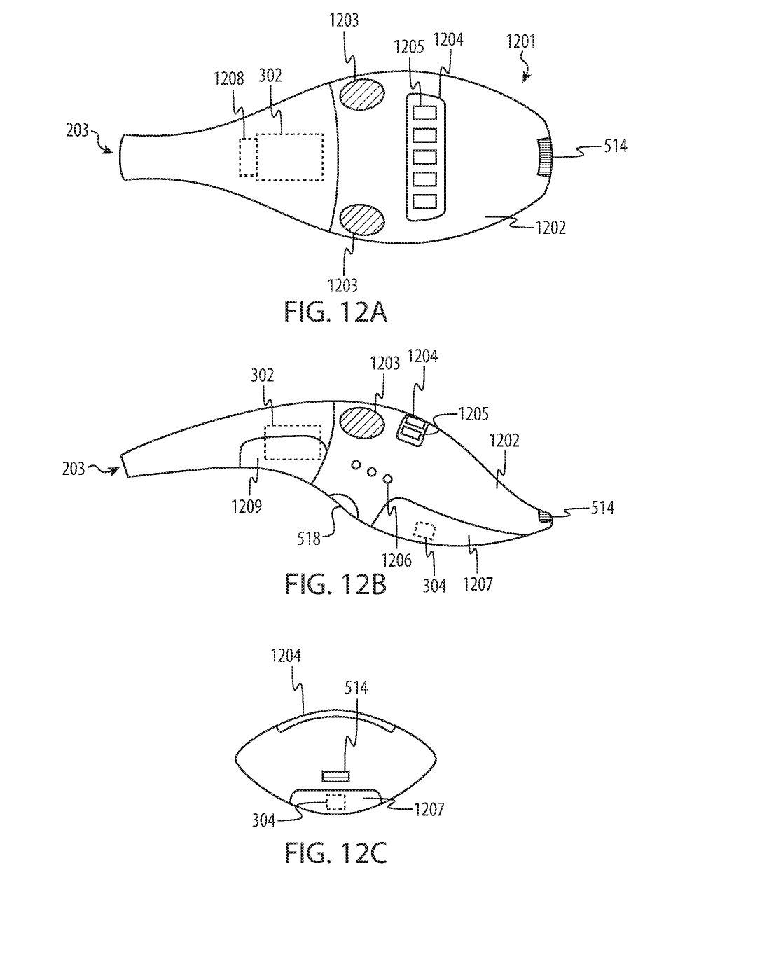

Referring to FIG. 12A a top perspective view of an alternate inhalation device 1201 is shown. Device 1201 includes an alternate external design but may incorporate some or all of the features and functions described herein. Device 1201 includes a housing 1202 which includes an integrated mouthpiece 203 as previously described. Device 1201 may also include one or more side switches 1203 which may be used for activation or selection of options in the device. Device 1202 may also include USB port 514 as previously described. A keypad 1204 may also include one or more touch keys 1205 for selecting various options on device 1202. Keypad 1204 may also include indicator 307 as previously described to inform the user of various functions and status of the device. Device 1201 contains removable or replaceable cartridge 302 containing the substance(s) to be inhaled. Cartridge 302 is secured in housing 1202 by a gasket 1208 or other sealing means to ensure secure attachment as discussed previously.

Referring to FIG. 12B, a side view of device 1201 is shown which includes mouth piece 203. Device 1201 may also include activation button 306 and a battery level indicator 1206. Alternatively indicator 1206 could be incorporated into display 1204 as previously described. Device 1201 may also include sensors and additional apparatus and devices shown and described herein. An activation button 518 as previously described may also be incorporated in lieu of or in addition to side switches 1203. A rechargeable battery 304 may be included inside the device housing with a battery compartment cover 1207 concealing the battery. The battery 304 may be replaceable or may be recharged through USB port 514 as previously described herein. Device 1201 also includes a removable cartridge cover 1209 which allows access to cartridge 302 in housing 1202. Removal of cover 1209 allows cartridge 302 to be removed and replaced. As discussed above, access to cartridge 302 may be restricted and thus removal of cover 1209 may require secured access through a password or other security device as discussed herein. Similarly, removal or cartridge 302 from housing 1202 may require such secured access as discussed herein. Replacement of cartridge 302 may include replacement of cartridges 302A and 302B as described herein.

Referring to FIG. 12C, a rear view of device 1201 is shown illustrating, side battery compartment cover 1207, USB port 514 and display 1204 all as shown placed on or in housing 1002.

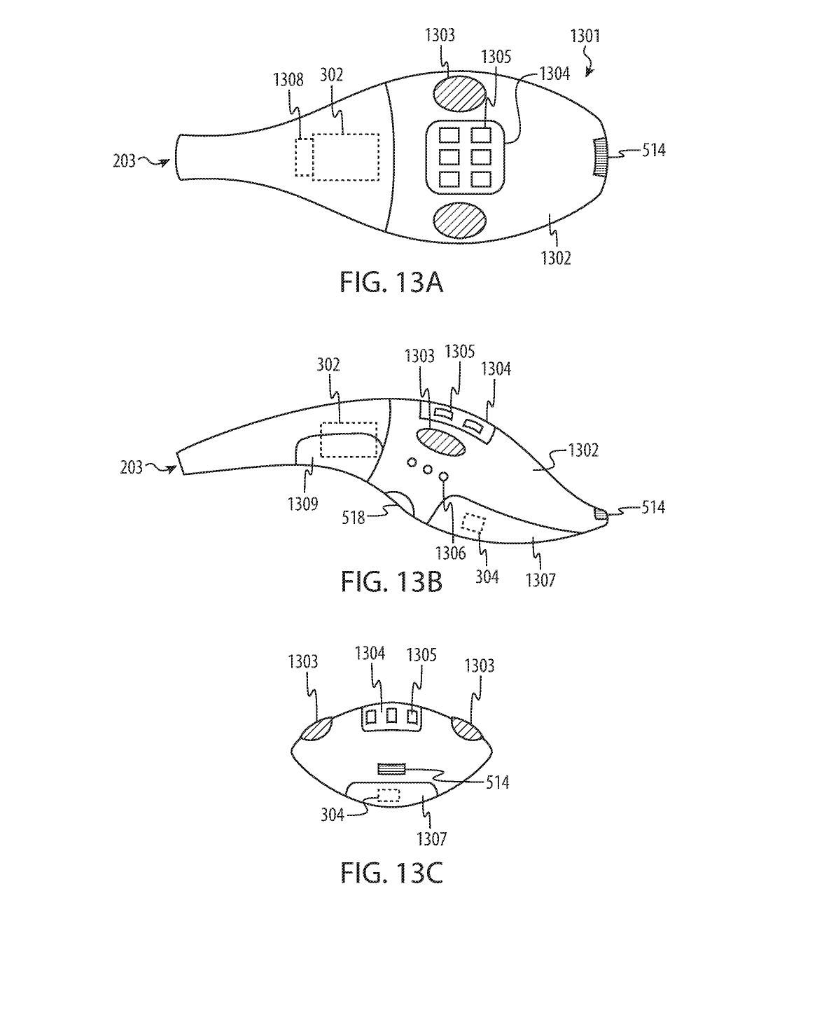

Referring to FIG. 13A a top perspective view of an alternate inhalation device 1301 is shown. Device 1301 includes an alternate external design but may incorporate some or all of the features and functions described herein. Device 1301 includes a housing 1302 which includes an integrated mouthpiece 203 as previously described. Device 1201 may also include one or more side switches 1303 which may be used for activation or selection of options in the device. Device 1302 may also include USB port 514 as previously described. A keypad 1304 may also include one or more touch keys 1305 for selecting various options on device 1302. Keypad 1304 may also include indicator 307 as previously described to inform the user of various functions and status of the device. Device 1301 contains removable or replaceable cartridge 302 containing the substance(s) to be inhaled. Cartridge 302 is secured in housing 1302 by a gasket 1308 or other sealing means to ensure secure attachment as discussed previously.

Referring to FIG. 13B, a side view of device 1301 is shown which includes mouth piece 203. Device 1301 may also include activation button 306 and a battery level indicator 1306. Alternatively indicator 1306 could be incorporated into display 1304 as previously described. Device 1301 may also include sensors and additional apparatus and devices shown and described herein. An activation button 518 as previously described may also be incorporated in lieu of or in addition to side switches 1303. A rechargeable battery 304 may be included inside the device housing with a battery compartment cover 1307 concealing the battery. The battery 304 may be replaceable or may be recharged through USB port 514 as previously described. Device 1301 also includes a removable cartridge cover 1309 which allows access to cartridge 302 in housing 1302. Removal of cover 1309 allows cartridge 302 to be removed and replaced. As discussed above, access to cartridge 302 may be restricted and thus removal of cover 1309 may require secured access through a password or other security device as discussed herein. Similarly, removal or cartridge 302 from housing 1302 may require such secured access as discussed herein. Replacement of cartridge 302 may include replacement of cartridges 302A and 302B as described herein.

Referring to FIG. 13C, a rear view of device 1301 is shown illustrating, side battery compartment cover 1307 USB port 514 and display 1304 all as shown placed on or in housing 1002.

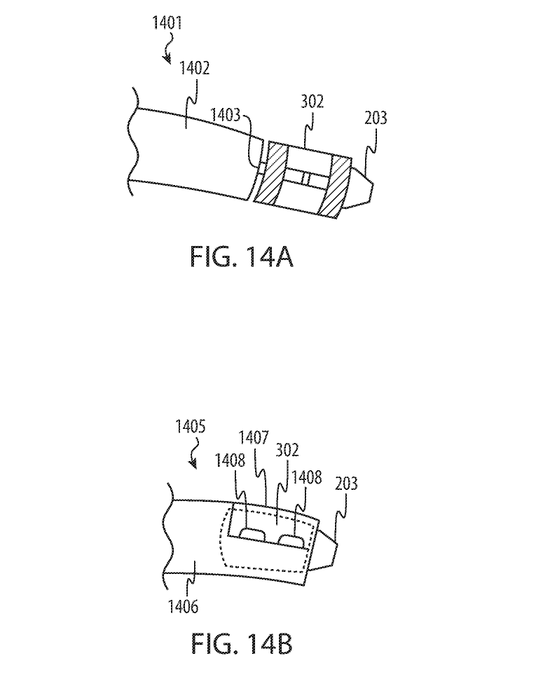

Referring to FIG. 14A, an alternate embodiment of an inhalation device 1401 is shown. In this embodiment, an atomization unit 1404 which may be similar to unit 712 described above, is attached to device body 1402 by threaded attachment 1403 in lieu of the pin attachment previously described with respect to unit 712. In this embodiment, mouthpiece 203 and cartridge tank 302 are removable and replaceable but may require secured access through a password or other security device as discussed herein. Replacement of cartridge 302 may include replacement of cartridges 302A and 302B as described herein.

Referring to FIG. 14B an alternate embodiment of an inhalation device 1405 is shown. In this embodiment, cartridge tank 302 is placed within device body 1406 adjacent to mouthpiece 203. Cartridge tank 302 is accessed through access cover 1407 which may be attached to device body 1406 at hinged connections 1408. In this embodiment, cartridge tank 302 is removable and replaceable but may require secured access through a password or other security device as discussed herein. That is, opening cover 1407 may require secured access and/or removal and replacement of cartridge 302 may require similar or additional secured access. Replacement of cartridge 302 may include replacement of cartridges 302A and 302B as described herein.

The monitoring devices and sensors referred to herein may be advantageously employed with device 201 (1001) depending upon intended use. For example, for opioid inhalation, certain sensors such as pulse oximetry and heart rate may be important to include to determine patient status while inhalation of antibiotics for example may not require such monitoring. Inhalation of anti-hypertensives, anti-arrythmics, antianginal drugs, anticonvulsants, Anxiolytics, antidepressants, antipsychotics, sedatives, hypnotics, opioids, NSAIDS, muscle relaxants, movement disorder drugs, antibiotics, bronchodilators, diabetes medications, thyroid medications, and antibiotics for pulmonary infections may dictate various combinations of sensors to effectively monitor a patient status as shown further in Table 1 and FIG. 9.

For conditions associated with the central nervous system such as Parkinsons disease, where anticonvulsants, anxiolytics, antidepressants, antipsychotics, sedatives, analgesics, anti-inflammatory, opioids and antipyretic drugs are used to treat such conditions, one or more health monitors such as an accelerometer, pulsoximeter, galvanic skin response, GPS locator for emergency medical response, and carbon dioxide monitor may be used. For administration of antibiotics, one or more of a pulse oximeter, carbon dioxide, galvanic skin response, temperature sensor, airflow sensor, and spirometer may be indicated. For administration of neuromuscular drugs one or more of a pulse oximeter, capnography airflow, blood pressure, electrodermal activity monitor, temperature sensor, electromyogram sensor, electrocardiogram, airflow sensor, skin response sensor, spirometer, glucometer, and accelerometer may be indicated, again as is shown in Table 1 and FIG. 9.

For treatment of respiratory conditions, where administration of bronchodilators, corticosteroids, antihistamines, anti-infectives and expectorants or cough suppressants are employed, monitors such as a pulse oximeter, carbon dioxide, galvanic skin monitor, temperature sensor, airflow sensor, spirometer, glucometer and a movement device such as an accelerometer may be indicated also as shown in Table 1 and FIG. 9.

While M-6-G are the preferred substances for use in the present system, dissolving any opioid/opioid derivative/intermediary/byproduct or other medication in a suitable medium which may include any vegetable oil (olive, coconut oils etc.)/triethylene glycol/propylene glycol at all ranges of temperatures and pressures may also be employed with the present system and, as such, are included in the definition of opioid composition as used herein. For example, dissolving any opiod/opioids in vegetable glycerin/propylene glycol/triethylene glycol, glycomorph, dhydroglycomorph, glycooxycodomorph/hydromorphoglyce/heroglyce etc. may be advantageously employed with the present embodiments.

As described herein, inhaled opioid compositions are administered with a safe inhalation delivery device 201 (1001). Opioid composition inhalation in a gaseous state is superior to available general anesthetic, effectively in an anesthesia enclosure 802, as shown in FIG. 8. In some embodiments flavoring or scents could be added to the opioid composition.

The system could be used to administer anti-hypertensives and antidepressants, a different class of drug but all amenable to the drug delivery system. In some embodiments any drugs on the US list of Schedule II drugs could be used. Some of these drugs are listed in Table 1. One advantage of the presently disclosed embodiments using pulmonary administration includes faster dispersion of medication to the patient. Opiates can be a bronchodilator and the onset of opiates is faster than oral ingestion giving faster pain relief. In addition, the delivery of inhaled opiates can be better controlled. These inhaled opiates are used with a safe delivery device 201. The pharmokinetics of inhaled opiates are nearly identical to the pharmokinetics of IV administered opiates.

M-6-G may be dissolved into glycerol/glycerin/propylene glycol or any suitable carrier wherein the carrier is miscible (dissolvable), with M-6-G. In one embodiment, M-6-G becomes metabolically active when heated between 40° C.-900° C. where the glycerol is “vaporized” for inhalation. Inhalation is a well-known drug delivery mechanism. No overdoses are likely to occur from vaping M-6-G such as M-6-G and there have been no recorded deaths from vaping opium. In some embodiments, patients can regulate their own dose. Excessive inhalation of M-6-G vapor (like smoking opium) will result in no more than a somniferous sleep. There is no respiratory depression associated with M-6-G as respiratory depression is a function of μ2 opioid receptors not activated by M-6-G.

M-6-G is an active metabolite of heroin and morphine and can be used to safely treat heroin addiction. M-6-G inhalation can safely reduce prescription pill abuse because there is no mu-2 activation to cause respiratory depression. M-6-G inhalation can safely be used to treat depression, anxiety, and insomnia in the same way smoking opium was used to treat these conditions historically. M-6-G inhalation can safely treat drug addiction utilizing the concept of harm reduction. M-6-G inhalation can be used as a psychiatric drug.

M-6-G can be mixed by pharmacist prescription with other naturally occurring alkaloids like caffeine, chocolate and even nicotine for harm reduction techniques. M-6-G has the same allergic potential as PG/VG but there can be no allergy to M-6-G as it is a naturally occurring endogenous opiate. M-6-G, unlike other opioids (including M-3-G), cannot produce constipation as none is metabolized in the gut. M-6-G, unlike other opiates (including M-3-G), can produce no itching as it is not associated with mast cell activation. M-6-G, unlike other opiates (including M-3-G), has no effect on the endocrine system and does not produce pituitary hormone secretion (delta, kappa & epsilon). M-6-G, unlike other opiates (including M-3-G), does not cause tolerance as tolerance is mediated by Nociceptin receptors. M-6-G inhalation is a potent bronchodilator and has can be used to treat pulmonary dysfunction, asthma, COPD, and other lung disorders.

M-6-G, unlike other opiates (including M-3-G), does not produce respiratory depression, miosis, euphoria, reduced GI motility that are mu-2 mediated. M-6-G, unlike other opiates (including M-3-G), does not produce Kappa and Sigma mediated side effects such as anxiety, depression, appetite suppression, convulsion, hallucinations, miosis, sedation, neuroprotection, dysphoria, or stress. M-6-G, unlike NSAIDS, does not produce peptic ulcer, or cardiac problems such as stroke and can be used as a harm reduction technology for NSAIDS. M-6-G does not cause withdrawal because physical dependence and withdrawal are mediated by mu-2 not present in M-6-G.

M-6-G inhalation is safer, cheaper and more effective than spinal fusion surgery for the treatment of back pain and is more effective than epidurals. M-6-G, unlike any other opiate, can be used safely in renal patients. M-6-G inhalation can be used for cocaine addiction in a harm reduction fashion. M-6-G inhalation can be used as a safe alternative to antidepressants, NSAIDS, aspirin, anti-rheumatoid drugs, and anxiety drugs. M-6-G inhalation can more safely and effectively treat painful neuropathies.

M-6-G inhalation can be used during the treatment of battlefield injuries as it requires no IV or injection whereby an injured soldier could use the inhaler to treat the pain before a medic can start an IV or in situations with no medic is available.

M-6-G inhalation can be used as a preoperative sedative in adults and children prior to painful procedure. M-6-G inhalation can be used in the hospital emergency department with little risk of sedation. M-6-G inhalation can be used as a general anesthetic because a better safety profile (an anesthesia vaping circuit) is included in the delivery device. M-6-G can be used preferably for acute pain as it does not cause physical dependence, and can be used as an anesthetic for postoperative pain and will not cause postoperative nausea and vomiting (PONV). M-6-G inhalation can be used as a replacement or adjuvant for muscle relaxation. M-6-G inhalation can be used to treat neuropathic pain states such as Chronic Regional Pain Syndrome (CRPS) more effectively than currently available medications. M-6-G inhalation is superior to available general anesthetic, effectively in an anesthesia vaporizer 802, as shown in FIG. 8.

Multiple indications for use of the for opioid composition usage in a novel heat-activated inhalation drug delivery device are described herein. Treatments using the opioid compositions in the pediatric population, or incapacitated adult population, may require an opioid composition neonatal or other opioid composition drug delivery tent such as that described herein.

Vaporized M-6-G can be used to treat aggressive behavior in prisoners. It can be used to subdue violent criminals using non-lethal force (e.g. by smoke grenade). Similarly, it has applications for SWAT teams in hostage situations. Vaporized M-6-G has military applications for subduing enemies for interrogation. Some studies have suggested that M-6-G such as M-6-G may lower violent tendencies via a pituitary mechanism. Some additional applications for inhaled opioid and other described substances herein administered by heat activated inhalation or other inhalation devices include: treatment of heroin addiction (in adults and children/babies); treatment of other substance addictions (including nicotine, alcohol, cocaine, crystal methamphetamine, and other drugs in adults and children/babies); treatment of major depressive disorder and acute depression episodes; treatment of anxiety, including all subtypes of anxiety (for example PTSD, social anxiety, general anxiety disorder, obsessive compulsive disorder, etc.); treatment of insomnia (including falling asleep, staying asleep and not waking early); treatment of respiratory disorders and diseases due to its bronchodilating effects (including pulmonary dysfunction, asthma, chronic obstructive pulmonary disorder, cystic fibrosis, and other lung disorders); treatment of acute pain (including battlefield injuries, arthritis, and back, replacing epidurals or spinal fusion surgery); treatment for menstrual cramps; treatment of neuropathic pain states (such as complex regional pain syndrome); use as a pre-operative sedative in adults in children to treat pain associated with the operation or procedure; use as a post-operative anesthetic for pain; use as a general anesthetic; treatment of erectile dysfunction; treatment for low sperm count; treatment for high blood pressure (acute or chronic); treatment for muscle relaxation as a replacement or adjuvant to other treatments; treatment for aggressive behaviors or in persons at high risk for developing aggressive behaviors; treatment for Parkinson's disease; treatment for urinary retention; treatment for closed angle glaucoma; and treatment of congestive heart-failure, pulmonary edema, hypertension and pulmonary bronchoconstriction.

M-6-G is similar to caffeine and chocolate in its harm profile and addiction profile and M-6-G can be used for crystal meth addiction treatment. M-6-G has no LD50 (lethal dose) and thus is safe for use by a patient without concern for an overdose. M-6-G is an anesthetic induction agent superior to propofol (in longer cases). In some embodiments flavoring or alkaloids such as caffeine, chocolate and nicotine could be added to or substituted for the M-6-G/glycerol mixture. Other prescription or non-prescription medications could be included or substituted for the M-6-G/glycerol mixture.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An inhalation apparatus comprising:
a power supply;
an activation unit connected to the power supply, the activation unit including:
a container of an opioid composition;
a carrier substance miscibly associated with the opioid composition;
a heating unit operatively connected to the container to vaporize a mixture of the carrier substance and the opioid composition;
a mouthpiece configured to receive the vaporized mixture from the heating unit;
a control unit associated with the activation unit to regulate an amount and or timing of the vaporized mixture delivered to
the mouthpiece; and
whereby the vaporized mixture is inhaled by a user through the mouthpiece.

2. The apparatus of claim 1, wherein the opioid composition includes one or more of the following: morphine, morphine sulfate, 6 monoacetylmorphine, morphine-6 glucuronide, morphine-6 glucuronide bromide, morphine-6 acetate, or morphine-6 sulfate, synthetic, natural or semi-synthetic opioid or other salt forms of these substances.

3. The apparatus of claim 1, wherein the container is a tamper proof container. including a substance to render the opioid composition unusable.

4. The apparatus of claim 1, wherein the apparatus further includes a user verification device including one or more selected from among: a fingerprint scanner; a retinal scanner; a combination lock; or a user password.

5. The apparatus of claim 1, further including a patient dosing safety feature wherein a pulse oximeter measures the patient's blood oxygen saturation.

6. The apparatus of claim 1, further including a device associated with the mouthpiece to contain exhaled gases from the user.

7. The apparatus of claim 6, wherein the device to contain includes a CO₂ scrubber or a soda lime trap.

8. A method for treating pain or addiction in a patient comprising the operations of:
activating an inhalation device;
mixing an opioid composition with a carrier substance;
heating the opioid composition and carrier substance mixture to an activation temperature; and
providing the activated opioid composition and carrier substance mixture to the patient through the inhalation device.

9. The method of claim 8, wherein the opioid concentrate, includes one or more of the following: morphine, morphine sulfate, 6 monoacetylmorphine, morphine-6 glucuronide, morphine-6 glucuronide bromide, and morphine-6 sulfate and other salt forms of these aforementioned substances.

10. The method of claim 8, wherein the vaporization temperature is about 50° C. to 250° C. or more.

11. The method of claim 8, wherein the carrier substance is selected from among propylene glycol vegetable oil, triethelyne glycol, glycerin, glycomorph, dhydroglycomorph, glycooxycodomorph, hydromorphoglyce, or heroglyce.

12. The method of claim 8, wherein the operation of activating includes authorizing a dose of opioid composition using one or more of fingerprint scan, pulse oximeter verification, health care provider authorization, retinal scan or password verification.

13. The method of claim 8, wherein the operation of activating includes notifying the patient that a dose of opioid composition is available by one or more of: visual, optical or haptic feedback.

14. The method of claim 8, wherein the step of providing activated opioid composition includes containing exhaled gases from the patient in a CO₂ scrubber or a soda lime trap.

15. A system for providing pain relief and for treating substance addiction comprising:
an inhalation apparatus containing an opioid composition;
a heating unit associated with the inhalation apparatus to heat the opioid composition to an activation temperature;
a wireless communication device operably associated with the inhalation apparatus to receive and transmit messages;
a control unit operably coupled with the inhalation apparatus, the control unit including:
a user verification device;
a device to monitor dosage and time intervals of usage of the inhalation apparatus; and
a device to indicate a status of one or more components of the inhalation apparatus and the opioid composition.

16. The system of claim 15, wherein the opioid composition includes one or more of the following:
morphine, morphine sulfate, 6 monoacetylmorphine, morphine-6 glucuronide, morphine-6 glucuronide bromide, and morphine-6 sulfate or other salt forms of these aforementioned substances.

17. The system of claim 15, wherein the user verification device is operably connected to the wireless communication device and includes one or more of: a fingerprint scanner; pulse oximeter; retinal scanner, biometric device, or password verification.

18. The system of claim 15, wherein the control unit may be programmable to:
verify user identity;
determine a user health status by correlating data from a pulse oximeter
associated with the inhalation apparatus with frequency of usage of the apparatus; and
provide an indication of the user health status.

19. The system of claim 18, wherein providing an indication includes at least one of:
sending a signal through the wireless communication device to notify a health care provider of the determined user health status; and
activating or deactivating the apparatus in response to the determined user health status.

20. The system of claim 15, wherein the inhalation apparatus includes a tent substantially enclosing the user.

21. A method for controlled delivery of opioid and other medications to a patient comprising the operations of:
activating an inhalation device;
mixing an opioid composition with a carrier substance; and
providing the activated opioid composition and carrier substance mixture to the patient through the inhalation device.

22. The method of claim 21, wherein the opioid includes one or more of the following: morphine, morphine sulfate, 6 monoacetylmorphine, morphine-6 glucuronide, morphine-6 glucuronide bromide, and morphine-6 sulfate, morphine-6-acetate and other salt forms of these aforementioned substances.

23. The method of claim 21 wherein the opioid and other medications are administered with a carrier substance selected from among propylene glycol vegetable oil, triethelyne glycol, glycerin, glycomorph, dhydroglycomorph, glycooxycodomorph, hydromorphoglyce, or heroglyce.

24. The method of claim 23, wherein the opioid and carrier substance are heated to a vaporization temperature of about 50° C. to 250° C. or more.

25. The method of claim 21, wherein the operation of activating includes authorizing a dose of opioid composition using one or more of fingerprint scan, pulse oximeter verification, health care provider authorization, retinal scan or password verification.

26. The method of claim 21, wherein the operation of activating includes notifying the patient that a dose of opioid composition is available by one or more of: visual, optical or haptic feedback.

27. The method of claim 21, wherein the step of providing activated opioid composition includes containing exhaled gases from the patient in a CO₂ scrubber or a soda lime trap.

28. The method of claim 21 wherein the medications include pharmaceutical compositions administered by inhalation as solution, suspension, aqueous solution, drops, irrigations, nebulized solution wherein solvent included water and all buffers, dry powder where the dry powder carrier includes lactose and other carriers, single-dose dry powder units, liquid soft mist wherein vehicle included water and other buffers, propellant-based solutions and suspensions wherein propellant includes all hydrofluoroalkanes, mucoadhesive solutions, and nasal rinses.

29. The method of claim 21 wherein the inhalation device includes pressurized meter dose inhalers, dry powder inhalers, breath-actuated dry powder inhalers, soft mist inhalers, jet nebulizers, ultrasonic nebulizers, vibrating mesh nebulizers, nasal spray bottles, intelligent inhalers, neti pots, intranasal mucosal atomization devices with or without syringes, intranasal vapor inhalers, squeeze bottles for oral and intranasal inhalation, gas driven spray atomizers, insufflators, spacers used with metered dose inhalers, and thermal vaporization aerosol devices.
